SEQUENCE LISTING (2 sequences)

SEQ ID NO: 1 - DNA, 20 bp, Artificial Sequence
  P5 adapter
aatgatacggcgaccaccga

SEQ ID NO: 2 - DNA, 24 bp, Artificial Sequence
  P7 adapter
caagcagaagacggcatacgagat

(19) United States
(12) Patent Application Publication
(10) Pub. No.: US 2023/0159983 A1
(43) Pub. Date: May 25, 2023

(54) METHOD FOR DETECTING ANALYTES OF VARYING ABUNDANCE

(71) Applicant: OLINK PROTEOMICS AB, Uppsala (SE)

(72) Inventors: John BROBERG, Uppsala (SE); Lotta WIK, Uppsala (SE); Martin LUNDBERG, Uppsala (SE); Niklas NORDBERG, Storvreta (SE)

(21) Appl. No.: 17/907,140

(22) PCT Filed: Mar. 26, 2021
(86) PCT No.: PCT/EP2021/058008

§ 371 Date: Sep. 23, 2022

(30) Foreign Application Priority Data
Mar. 27, 2020 (GB) 2004472.3
Mar. 27, 2020 (GB) 2004474.9
Mar. 27, 2020 (GB) 2004484.8

(51) Int. Cl. C12Q 1/6804; C12Q 1/6806; C12Q 1/6837

(52) U.S. Cl. CPC C12Q 1/6804; C12Q 1/6806; C12Q 1/6837; C12Q 2600/16; C12Q 2600/166

(57) ABSTRACT

The present invention provides a method of detecting multiple analytes in a sample, wherein said analytes have varying levels of abundance in the sample, said method comprising: (i) providing multiple aliquots from the sample; and (ii) in each aliquot, detecting a different subset of the analytes by performing a separate multiplex assay for each aliquot, wherein the analytes in each subset are selected based on their predicted abundance in the sample.

### FIELD

The present invention provides a method of detecting multiple analytes in a sample, wherein the analytes have varying levels of abundance in the sample. In the method, multiple aliquots of the sample are provided, and in each aliquot a subset of the analytes is detected, the subset of analytes being selected based on their predicted abundance in the sample. Also provided is a method of detecting an analyte in a sample, wherein the analyte is detected by detecting a reporter nucleic acid molecule specific for the analyte. In this method a PCR reaction is performed to amplify the reporter nucleic acid molecule, in which PCR an internal control is used. The methods of the invention find particular utility in the context of a proximity extension assay (PEA).

### BACKGROUND

Modern proteomics methods require the ability to detect a large number of different proteins (or protein complexes) in a small sample volume. To achieve this, multiplex analysis must be performed. Common methods by which multiplex detection of proteins in a sample may be achieved include proximity extension assays (PEA) and proximity ligation assays (PLA). PEA and PLA are described in WO 01/61037; PEA is further described in WO 03/044231, WO 2004/094456, WO 2005/123963, WO 2006/137932 and WO 2013/113699. However, when, as is common, the proteins of interest are present in a wide concentration range, this presents a challenge, since the signal from proteins of high concentration may overwhelm the signal from proteins of low concentration, resulting in a failure to detect proteins present at lower concentrations.

The present invention provides detection methods whereby analytes (e.g. proteins) present in a sample at a wide range of concentrations may be reliably detected, improving the accuracy of multiplex detection methods. The methods of the invention may be applied to PEA or PLA as mentioned above, but may also be applied to any other technique used in multiplex analyte detection.

PEA and PLA are proximity assays, which rely on the principle of “proximity probing”. In these methods an analyte is detected by the binding of multiple (i.e. two or more, generally two or three) probes, which when brought into proximity by binding to the analyte (hence “proximity probes”) allow a signal to be generated. Typically, at least one of the proximity probes comprises a nucleic acid domain (or moiety) linked to the analyte-binding domain (or moiety) of the probe, and generation of the signal involves an interaction between the nucleic acid moieties and/or a further functional moiety which is carried by the other probe(s). Thus signal generation is dependent on an interaction between the probes (more particularly between the nucleic acid or other functional moieties/domains carried by them) and hence only occurs when the necessary probes have bound to the analyte, thereby lending improved specificity to the detection system.

In PEA, nucleic acid moieties linked to the analyte-binding domains of a probe pair hybridise to one another when the probes are in close proximity (i.e. when bound to a target), and are then extended using a nucleic acid polymerase. The extension product forms a reporter nucleic acid, detection of which demonstrates the presence in a sample of interest of a particular analyte (the analyte bound by the relevant probe pair). In PLA, nucleic acid moieties linked to the analyte-binding domains of a probe pair come into proximity when the probes of the probe pair bind their target, and may be ligated together, or alternatively they may together template the ligation of separately added oligonucleotides which are able to hybridise to the nucleic acid domains when they are in proximity. The ligation product is then amplified, acting as a reporter nucleic acid. Multiplex analyte detection using PEA or PLA may be achieved by including a unique barcode sequence in the nucleic acid moiety of each probe. A reporter nucleic acid molecule corresponding to a particular analyte may be identified by the barcode sequences it contains. The methods of the present invention find particular utility in multiplex PEA and PLA methods.

The methods of the invention may be of utility in at least any field in which proteomics is used, in particular in diagnostics in the context of biomarker identification and quantification. Modern personalised medicine requires the ability to assess large panels of biomarkers, e.g. in the field of oncology. As personalised medicine becomes ever more widespread, the ability to accurately identify and quantify a large number of biomarkers in a sample (across a range of concentrations) is increasing in importance. This need is addressed by the present invention.

### SUMMARY OF INVENTION

To this end, in a first aspect the present invention provides a method of detecting multiple analytes in a sample, wherein said analytes have varying levels of abundance in the sample, said method comprising:

(i) providing multiple aliquots from the sample; and

(ii) in each aliquot, detecting a different subset of the analytes by performing a separate multiplex assay for each aliquot, wherein the analytes in each subset are selected based on their predicted abundance in the sample.

In a second aspect, the invention provides a method of detecting an analyte in a sample, wherein the analyte is detected by detecting a reporter nucleic acid molecule specific for the analyte, said method comprising performing a PCR reaction to generate a PCR product of the reporter nucleic acid molecule and detecting said PCR product;

wherein an internal control is provided for the PCR reaction, and said internal control is:

(i) a separate component which is present in a pre-determined amount, and which is, or comprises, or leads to the generation of, a control nucleic acid molecule which is amplified by the same primers as the reporter nucleic acid molecules; and/or

(ii) a unique molecular identifier (UMI) sequence present in each reporter nucleic acid molecule and/or in each control nucleic acid molecule, which is unique to each molecule.

In a third aspect, the invention provides a method of detecting an analyte in a sample, wherein the analyte is detected by detecting a reporter nucleic acid molecule for the analyte, said method comprising performing a PCR reaction to generate a PCR product of the reporter nucleic acid molecule and detecting said PCR product, wherein an internal control is included in the PCR reaction and said internal control is present in a pre-determined amount and is, or comprises, or leads to the generation of, a control nucleic acid molecule wherein the control nucleic acid molecule comprises a sequence which is the reverse sequence of the reporter nucleic acid molecule.

### DETAILED DESCRIPTION

As detailed above, the first aspect of the present invention provides a method for detecting multiple analytes in a sample, wherein the analytes have varying levels of abundance in the sample. The method relies on performing separate sets of assays grouped according to the abundance of the analytes to be assayed.

Accordingly, alternatively viewed, the method as disclosed herein may be defined as a method of detecting multiple analytes in a sample, wherein said analytes have varying levels of abundance in the sample, said method comprising:

performing a separate block of assays on each of separate multiple aliquots from said sample, to detect in each separate aliquot a subset of the analytes, wherein the analytes in each subset are selected based on their predicted abundance in the sample.

Each block of assays performed on an individual aliquot is accordingly a multiplex assay. The multiplex assay to detect multiple analytes in the analyte subset (i.e. the analyte subset designated to be detected in any one particular aliquot) may thus be viewed as an “abundance block”. The term “abundance block” as used herein thus refers to a block of assays (or set of assays) performed to detect a particular group, or subset, of the analytes to be detected (i.e. assayed for) in the sample, wherein the analytes are assigned to each block (or set) of assays based on their abundance in the sample, namely their expected or predicted abundance, or relative abundance in the sample. In other words, the assays are grouped, or “blocked” based on abundance. Thus, different aliquots, or different abundance blocks, may be designated for the detection of a particular subset of analytes, based on, for example, low, high or varying degrees of intermediate levels of abundance etc.. This does not imply that the abundance of each analyte in a block, or set of assays is the same or about the same; the abundance may vary between different analytes/assays in the block or set, and/or between different samples.

The term “analyte” as used herein (in respect of all aspects of the present invention) means any substance (e.g. molecule) or entity it is desired to detect by the method of the invention. The analyte is thus the “target” of the assay method of the invention, i.e. the substance detected or screened for using the method of the invention.

The analyte may accordingly be any biomolecule or chemical compound it is desired to detect, for example a peptide or protein, or a nucleic acid molecule or a small molecule, including organic and inorganic molecules. The analyte may be a cell or a microorganism, including a virus, or a fragment or product thereof. It will be seen therefore that the analyte can be any substance or entity for which a specific binding partner (e.g. an affinity binding partner) can be developed. All that is required is that the analyte is capable of simultaneously binding at least two binding partners (more particularly, the analyte-binding domains of at least two proximity probes).

Proximity probe-based assays have found particular utility in the detection of proteins or polypeptides. Analytes of particular interest thus include proteinaceous molecules such as peptides, polypeptides, proteins or prions or any molecule which includes a protein or polypeptide component, etc., or fragments thereof. In a particularly preferred embodiment of the invention, the analyte is a wholly or partially proteinaceous molecule, most particularly a protein. That is to say, it is preferred that the analyte is or comprises a protein.

The analyte may be a single molecule or a complex that contains two or more molecular subunits, which may or may not be covalently bound to one another, and which may be the same or different. Thus in addition to cells or microorganisms, such a complex analyte may also be a protein complex, or a biomolecular complex comprising a protein and one or more other types of biomolecule. Such a complex may thus be a homo- or hetero-multimer. Aggregates of molecules e.g. proteins may also be target analytes, for example aggregates of the same protein or different proteins. The analyte may also be a complex between proteins or peptides and nucleic acid molecules such as DNA or RNA. Of particular interest may be the interactions between proteins and nucleic acids, e.g. regulatory factors, such as transcription factors, and DNA or RNA. Thus in a particular embodiment the analyte is a protein-nucleic acid complex (e.g. a protein-DNA complex or a protein-RNA complex). In another embodiment, the analyte is a non-nucleic acid analyte, by which is meant an analyte which does not comprise a nucleic acid molecule. Non-nucleic acid analytes include proteins and protein complexes, as mentioned above, small molecules and lipids.

The method of the invention is directed to detecting multiple analytes in a sample. The multiple analytes may be of the same type (e.g. all the analytes may be proteins, or protein complexes), or of different types (e.g. some analytes may be proteins, others protein complexes, others lipids, others protein-DNA or protein-RNA complexes, etc., or any combination of such types of analytes).

The term “multiple” as used in the present disclosure means more than one (that is to say, two or more), in line with its standard definition. However, the method of the first aspect of the invention requires separate multiplex reactions to be performed for multiple (i.e. at least two) aliquots of a sample. As used herein, the term “multiplex” is used to refer to an assay in which multiple (i.e. at least two) different analytes are assayed at the same time, and more particularly in the same aliquot of the sample, or in the same reaction mixture. Thus it is apparent that the minimum number of analytes to be detected according to the method of the first aspect of the present invention is four (two analytes to be detected in each of two aliquots of sample). However, it is preferred that considerably more analytes than four are detected according to the present method. Preferably at least 10, 20, 50, 100, 200, 300, 400, 500, 600, 700, 800, 900, 1000, 1100, 1200, 1300, 1400 or 1500 or more analytes are detected according to the present method.

The term “detecting” or “detected” is used broadly herein to include any means of determining the presence or absence of an analyte (i.e. determining whether a target analyte is present in a sample of interest or not). Accordingly, if a method of the invention is performed and an attempt is made to detect a particular analyte of interest in a sample, but the analyte is not detected because it is not present in the sample, the step of “detecting the analyte” has still been performed, because its presence or absence from the sample has been assessed. The step of “detecting” an analyte is not dependent on that detection proving successful, i.e. on the analyte actually being detected.

Detecting an analyte may further include any form of measurement of the concentration or abundance of the analyte in the sample. Either the absolute concentration of a target analyte may be determined, or a relative concentration of the analyte, for which purpose the concentration of the target analyte may be compared to the concentration of another target analyte (or other target analytes) in the sample or in other samples.

Thus “detecting” may include determining, measuring, assessing or assaying the presence or absence or amount of an analyte in any way. Quantitative and qualitative determinations, measurements or assessments are included, including semi-quantitative determinations. Such determinations, measurements or assessments may be relative, for example when two or more different analytes in a sample are being detected, or absolute. As such, the term “quantifying” when used in the context of quantifying a target analyte in a sample can refer to absolute or to relative quantification. Absolute quantification may be accomplished by inclusion of known concentration(s) of one or more control analytes and/or referencing the detected level of the target analyte with known control analytes (e.g. through generation of a standard curve). Alternatively, relative quantification can be accomplished by comparison of detected levels or amounts between two or more different target analytes to provide a relative quantification of each of the two or more different analytes, i.e. relative to each other. Methods by which quantification can be achieved in the method of the invention are discussed further below.

The method of the invention is for detecting multiple analytes in a sample. Any sample of interest may be assayed according to the invention. That is to say any sample which contains or may contain analytes of interest, and which a person wishes to analyse to determine whether or not it contains analytes of interest, and/or to determine the concentrations of analytes of interest therein.

Any biological or clinical sample may thus be analysed according to the present invention, e.g. any cell or tissue sample of or from an organism, or any body fluid or preparation derived therefrom, as well as samples such as cell cultures, cell preparations, cell lysates etc. Environmental samples, e.g. soil and water samples, or food samples may also be analysed according to the invention. The samples may be freshly prepared or they may be prior-treated in any convenient way e.g. for storage.

Representative samples thus include any material which may contain a biomolecule, or any other desired or target analyte, including for example foods and allied products, clinical and environmental samples. The sample may be a biological sample, which may contain any viral or cellular material, including prokaryotic or eukaryotic cells, viruses, bacteriophages, mycoplasmas, protoplasts and organelles. Such biological material may thus comprise any type of mammalian and/or non-mammalian animal cell, plant cells, algae including blue-green algae, fungi, bacteria, protozoa etc.

It is preferred that the sample is a clinical sample, for instance whole blood and blood-derived products such as plasma, serum, buffy coat and blood cells, urine, faeces, cerebrospinal fluid or any other body fluid (e.g. respiratory secretions, saliva, milk etc.), tissues and biopsies. It is particularly preferred that the sample is a plasma or serum sample. Thus the method of the invention may be used in the detection of biomarkers, for instance, or to assay a sample for pathogen-derived analytes. The sample may in particular be derived from a human, though the method of the invention may equally be applied to samples derived from non-human animals (i.e. veterinary samples). The sample may be pre-treated in any convenient or desired way to prepare it for use in the method of the invention, for example by cell lysis or removal, etc.

The method of the first aspect of the invention is for detecting multiple analytes in a sample wherein the analytes have varying levels of abundance in the sample. That is to say, the analytes are present in the sample at different concentrations, or at a range of concentrations. It is not required that every analyte in the sample is present at a substantially different concentration to every other analyte, but rather that not all analytes are present at substantially the same concentration. Although the analytes in the sample are present at a range of concentrations, it may be that certain analytes are present at very similar concentrations.

It may be that the analytes are present in the sample over a concentration range that spans several orders of magnitude. For instance, it may be that the analyte(s) present (or expected to be present) in the sample at the highest concentration are present (or expected to be present) at a concentration about 1000-fold higher than the (expected) concentration of the analyte (expected to be) present at the lowest concentration in the sample. Analytes in the sample may, for instance, vary in concentration relative to each other about 10-fold, about 100-fold, about 1000-fold or more, and of course any value in between. In a clinical sample, analytes may be present across a range of several orders of magnitude, e.g. 3, 4, 5 or 6 or more orders of magnitude.

The level or value for the abundance which is used to block or group together different analytes, or more particularly the assays for different analytes, may not be dependent only on the absolute level or concentration of the analyte present in the sample (or expected to be present). Other factors may be considered, including the nature of the assay method, differences in performance of the assay for different analytes, etc. For example, in the case of detection assays based on antibodies or other binding agents, this may depend on antibody affinity for the analyte, or avidity etc. Such variability between assays for different analytes may be taken into account. For example the abundance may reflect the abundance of analyte that is detected in the assay, in terms of the assay output value or measurement. Accordingly, the predicted abundance on the basis of which analytes in a subset are selected may depend at least on the predicted level or concentration of the analyte in a sample, but it may also or alternatively depend on the predicted level of or value for abundance to be determined in a particular detection assay. Put another way, the abundance of an analyte in the sample may be its apparent abundance, or a notional abundance which depends on the detection assay. The apparent abundance of an analyte may vary depending on the assay used, and in particular the sensitivity of that assay.

The method comprises providing multiple (that is to say, at least two) aliquots from the sample. That is to say, multiple separate portions of the sample are provided. The sample may be divided into multiple aliquots (such that the entire sample is aliquoted) or some of the sample may be provided as aliquots, without using the entire sample. The aliquots may be of the same size, or volume, or of different sizes, or volumes, or some aliquots may be of the same size and others of different sizes.

At least some of the aliquots may be diluted. For instance, samples may be diluted 1:2, 1:4, 1:5, 1:10, etc. In particular, aliquots may be subjected to 10-fold dilutions, i.e. one or more aliquots may be diluted 10-fold (or 1:10), one or more aliquots may be diluted 100-fold (1:100), and one or more aliquots may be diluted 1000-fold (1:1000). If desired, further dilutions may be made (e.g. 1:10,000 or 1:100,000), though as a rule a maximum dilution of 1:1000 can be expected to suffice. One or more aliquots may be undiluted (referred to herein as 1:1).

In a particular embodiment, a series of 10-fold dilutions is made, providing aliquots with the following dilutions: 1:1, 1:10, 1:100 and 1:1000. In this embodiment, the 1:10 dilution is generated by making a 10-fold dilution of the undiluted sample. The 1:100 and 1:1000 dilutions may be made by making direct 100-fold and 1000-fold dilutions (respectively) of the undiluted sample, or by making serial 10-fold dilutions of the 1:10 diluted aliquot (i.e. the 1:10 diluted aliquot may be diluted 10-fold to yield the 1:100 diluted aliquot, and the 1:100 diluted aliquot diluted 10-fold to yield the 1:1000 diluted aliquot). Sample dilutions (and indeed all pipetting steps throughout the methods of the invention) may be performed manually, or alternatively using an automated pipetting robot (such as an SPT Labtech Mosquito).

Dilutions of the sample may be made with any suitable diluent, which may depend on the type of sample being assayed. For instance, the diluent may be water or saline solution, or a buffer solution, in particular a buffer solution comprising a biologically-compatible buffer compound (i.e. a buffer compatible with the detection assay used, for instance a buffer compatible with a PEA or PLA). Examples of suitable buffer compounds include HEPES, Tris (i.e. Tris(hydroxymethyl)aminomethane), disodium phosphate, etc. Suitable buffers for use as diluent include PBS (phosphate-buffered saline), TBS (Tris-buffered saline), HBS (HEPES-buffered saline), etc. The buffer (or other diluent) used must be made up in a purified solvent (e.g. water) such that it does not contain contaminant analytes. The diluent should thus be sterile, and if water is used as diluent or the base of the diluent, the water used is preferably ultrapure (e.g. Milli-Q water).

Any suitable number of aliquots may be provided from the sample. As noted above, at least two aliquots are provided, though in most embodiments more than two will be provided. In a particular embodiment, as detailed above, four aliquots may be provided: an undiluted sample aliquot and aliquots in which the sample is diluted 1:10, 1:100 and 1:1000. More or fewer aliquots than this may be provided, if more or fewer sample dilutions are desired. Moreover, one or more aliquots of each dilution factor may be provided, in accordance with the desires/requirements of the particular assay performed.

Once the multiple aliquots have been provided from the sample, a separate multiplex assay is performed for each aliquot, in order to detect a subset of the target analytes in each aliquot. A separate multiplex assay is performed for each aliquot, such that each aliquot is analysed separately (i.e. the multiple aliquots are not mixed during the multiplex reactions). Across all the aliquots provided, and upon which multiplex assays are performed, all the target analytes are detected. That is to say, across all the aliquots, assays are performed to determine whether each target analyte is present in or absent from the sample of interest. However, each individual assay to detect a particular analyte may be performed in only one aliquot. Thus different subsets of analytes are detected in each aliquot, in other words different analytes are detected in each aliquot. Preferably, the subsets detected in each aliquot are wholly different, i.e. each target analyte is detected in only one aliquot, such that there is no overlap between analyte subsets. However, in some embodiments particular analytes may be detected in multiple aliquots, if deemed appropriate. In this instance there would be some overlap of analytes between the subsets, in that some analytes would be present in multiple analyte subsets, but other analytes would be present in only one subset.

The analytes in each subset are selected based on their predicted abundance (i.e. concentration) in the sample. That is to say, analytes which may be expected to be present in the sample at a similar concentration may be included in the same subset, and analysed in the same multiplex reaction. Conversely, analytes which may be expected to be present in the sample at different concentrations may be included in different subsets, and analysed in different multiplex reactions. Each analyte is assigned to a subset of analytes which are expected to be present at a similar concentration (e.g. a concentration within a particular order of magnitude) in the sample. Each subset of analytes is then detected in a sample aliquot which is diluted by an appropriate factor in view of the expected concentrations of the analytes. Thus analytes expected to be present at the lowest concentrations may be detected in an undiluted aliquot, or an aliquot having a low dilution factor; analytes expected to be present at the highest concentrations are detected in the most diluted aliquot; and analytes expected to be present at concentrations in between these extremes are detected in aliquots having “in-between” dilution factors.

As noted above, in some embodiments certain analytes may be included in multiple subsets. This may for instance be the case if an analyte has an expected concentration essentially in between the expected concentrations of two subsets, such that it does not clearly “belong” to either of them. In this instance, the analyte may be included in both subsets. An analyte might also be included in two (or more) subsets if it is known that the analyte could be present in the sample in an unusually wide range of concentrations.

It will be appreciated that given that the analytes in each subset are selected based on their predicted abundance in the sample, there may be different numbers of analytes in each subset. Alternatively there may be the same number of analytes in each subset, as appropriate.

The abundance/concentration of each analyte in the sample may be predicted based on known facts regarding the normal level of each analyte in the sample type to be analysed. For instance, if the sample is a plasma or serum sample (or a sample of any other bodily fluid), the concentration of the analytes therein may be predicted based on the known concentrations of species in these fluids. Normal plasma concentrations of a wide range of analytes of potential interest are available from https://www.olink.com/resources-support/document-download-center/. However, as noted above, the abundance value used to allocate an analyte to a particular subset (block) can depend on the assay, and the results (e.g. measurements) which are obtainable from that assay.

As detailed above, a multiplex reaction is performed on each aliquot, to detect all the analytes in the subset which are to be analysed in the aliquot. As noted, the term “multiplex” means an assay in which at least two different analytes are assayed at the same time. Preferably however considerably more than two analytes are assayed in each multiplex reaction. For instance, each multiplex reaction may assay at least 5, 10, 15, 20, 25, 30, 40, 50, 60 analytes or more. Certain multiplex reactions may assay more than this number of analytes, e.g. at least 70, 80, 90, 100, 110, 120, 130, 140 or 150 analytes or more.

In a particular embodiment of this aspect of the invention, in each aliquot the analytes are detected by detecting a reporter nucleic acid molecule specific for each analyte. In this embodiment, the presence of a particular analyte in the sample results in the production during the detection assay of a nucleic acid molecule with a particular nucleotide sequence, which is known to correspond to the particular analyte. Detection of the particular nucleotide sequence indicates that the analyte to which the sequence corresponds is present in the sample. A “reporter nucleic acid molecule” is thus a nucleic acid molecule whose synthesis during the detection assay indicates the presence in the sample of a particular analyte. The reporter nucleic acid molecule may be an RNA molecule or a DNA molecule. Preferably it is a DNA molecule.

The reporter nucleic acid molecule may be generated by any means known in detection assays of the art. For instance, it may be generated by ligation of two (or more) nucleic acids to each other, forming a unique nucleotide sequence indicative of the presence of the analyte in the sample. Alternatively, the reporter nucleic acid molecule may be generated by extension of a provided nucleic acid molecule along a template nucleic acid molecule. Combinations of extensions and ligations may also be used.

Reporter nucleic acid molecules are thus generated during the multiplex detection assays performed for each aliquot. To generate a reporter nucleic acid molecule, any detection assay which acts by generation of such nucleic acid molecules may be used. In a particular embodiment, the reporter nucleic acid molecule is generated in the context of a proximity extension assay (PEA). That is to say, a multiplex PEA may be performed in order to detect the analytes in each aliquot, and thus in the sample. In another embodiment, the reporter nucleic acid molecule is generated in the context of a proximity ligation assay (PLA), i.e. a multiplex PLA may be performed in order to detect the analytes in each aliquot. Methods for performing PEAs and PLAs are known in the art, as described above. It is particularly preferred that the detection assay performed is a PEA.

Following generation of the reporter nucleic acid molecule, it is preferably amplified for ease of detection. Amplification of the reporter nucleic acid molecule is preferably performed by PCR, though any other method of nucleic acid amplification may be utilised, e.g. loop-mediated isothermal amplification (LAMP).

As noted above, each reporter nucleic acid molecule is specific for a particular analyte. Thus, a reporter nucleic acid molecule identifies a given analyte, or more particularly, may contain a sequence or domain which functions as an identification (ID) sequence, or tag, by which an analyte may be detected. The ID sequence may be detected for example by serving as a binding site for probes or primers etc., as detailed further below, or more directly by sequencing. Accordingly, alternatively expressed, this specificity may be achieved by the presence in the reporter nucleic acid molecule of one or more barcode sequences. Broadly speaking, a barcode sequence may be defined as a nucleotide sequence within the reporter nucleic acid molecule which identifies the reporter, and thus the detected analyte. It may be that the entirety of each reporter nucleic acid molecule generated in the detection assays is unique, in which case the entire reporter nucleic acid molecule may be considered a barcode sequence. More commonly, one or more smaller sections of the reporter nucleic acid molecule act as barcode sequences.

The analytes in the sample are detected by detection of the specific barcode sequences within the reporter nucleic acid molecules generated during the multiplex detection assay. This may be achieved in a number of ways. Firstly, specific barcode sequences may be detected by sequencing of all the reporter nucleic acid molecules generated during the multiplex detection assay. By sequencing all reporter nucleic acid molecules generated, all the different reporter nucleic acid molecules generated may be identified by their barcode sequences, and thus all the analytes present in the sample may be identified (based on whether the reporter nucleic acid molecule known to correspond to each target analyte is detected or not). Nucleic acid sequencing is the preferred method of reporter nucleic acid detection/analysis.

Other suitable methods for detecting the reporter nucleic acid molecule include PCR-based methods. For instance, quantitative PCR utilising “TaqMan” probes may be performed. In this instance, the reporter nucleic acid molecules (or at least a section of each reporter nucleic acid molecule comprising the barcode sequence) is amplified, and a probe complementary to each barcode sequence is provided, with each different probe being conjugated to a different, distinguishable fluorophore. The presence or absence of each barcode (and thus reporter nucleic acid molecule, and thus analyte) can then be determined based on whether the particular barcode is amplified. However, it is apparent that PCR-based methods such as described above are only suitable for analysis of relatively small numbers of different sequences at the same time, although combinatorial methods using probes for decoding barcode sequences are known and may be used to extend multiplexing capacity to a degree. Nucleic acid sequencing does not have any real limit on the number of sequences which can be identified in any one go, enabling higher levels of multiplex reaction than detection using PCR, hence sequencing is the preferred method for reporter nucleic acid molecule detection.

Preferably, a form of high throughput DNA sequencing is used to detect the reporter nucleic acid molecules. Sequencing by synthesis is the preferred DNA sequencing method. Examples of sequencing by synthesis techniques include pyrosequencing, reversible dye terminator sequencing and ion torrent sequencing, any of which may be utilised in the present method. Preferably the reporter nucleic acids are sequenced using massively parallel DNA sequencing. Massively parallel DNA sequencing may in particular be applied to sequencing by synthesis (e.g. reversible dye terminator sequencing, pyrosequencing or ion torrent sequencing, as mentioned above). Massively parallel DNA sequencing using the reversible dye terminator method is a preferred sequencing method. Massively parallel DNA sequencing using the reversible dye terminator method may be performed, for instance, using an Illumina® NovaSeq™ system.

As is known in the art, massively parallel DNA sequencing is a technique in which multiple (e.g. thousands or millions or more) DNA strands are sequenced in parallel, i.e. at the same time. Massively parallel DNA sequencing requires target DNA molecules to be immobilised to a solid surface, e.g. to the surface of a flow cell or to a bead. Each immobilised DNA molecule is then individually sequenced. Generally, massively parallel DNA sequencing employing reversible dye terminator sequencing utilises a flow cell as the immobilisation surface, and massively parallel DNA sequencing employing pyrosequencing or ion torrent sequencing utilises a bead as the immobilisation surface.

As is known to the skilled person, immobilisation of DNA molecules to a surface in the context of massively parallel sequencing is generally achieved by the attachment of one or more sequencing adapters to the ends of the molecules. The method of the invention may thus include the addition of one or more adapters for sequencing (sequencing adapters) to the reporter nucleic acid molecules.

Commonly, the sequencing adapters are nucleic acid molecules (in particular DNA molecules). In this instance, short oligonucleotides complementary to the adapter sequences are conjugated to the immobilisation surface (e.g. the surface of the bead or flow cell) to enable annealing of the target DNA molecules to the surface, via the adapter sequences. Alternatively, any other pair of binding partners may be used to conjugate the target DNA molecule to the immobilisation surface, e.g. biotin and avidin/streptavidin. In this case biotin may be used as the sequencing adapter, and avidin or streptavidin conjugated to the immobilisation surface to bind the biotin sequencing adapter, or vice versa.

Sequencing adapters may thus be short oligonucleotides (preferably DNA), generally 10-30 nucleotides long (e.g. 15-25 or 20-25 nucleotides long). As detailed above, the purpose of a sequencing adapter is to enable annealing of the target DNA molecules to an immobilisation surface, and accordingly the nucleotide sequence of a nucleic acid adaptor is determined by the sequence of its binding partner conjugated to the immobilisation surface. Aside from this, there is no particular constraint on the nucleotide sequence of a nucleic acid sequencing adaptor.

A sequencing adapter may be added to a reporter nucleic acid molecule of the invention during PCR amplification. In the case of a nucleic acid sequencing adapter this can be achieved by including a sequencing adapter nucleotide within in one or both primers. Alternatively, if the sequencing adaptor is a non-nucleic acid sequencing adaptor (e.g. a protein/peptide or small molecule) an adapter may be conjugated to one or both PCR primers. Alternatively, a sequencing adapter may be attached to a reporter nucleic acid molecule by directly ligating or conjugating the sequencing adapter to the reporter nucleic acid molecule. Preferably the one or more sequencing adapters used in the present method are nucleic acid sequencing adapters.

One or more nucleic acid sequencing adapters may be added to the reporter molecule in one or more ligation and/or amplification steps. Thus if, for instance, two sequencing adapters are added to the reporter nucleic acid molecule (one at each end), these may be added in a single step (e.g. by PCR amplification using a pair of primers which both contain a sequencing adapter) or in two steps. The two steps may be performed using the same or different methods, e.g. a first sequencing adapter may be added to the reporter nucleic acid molecule by ligation and the second by PCR amplification, or vice versa; or a first amplification reaction may be performed to add a first sequencing adapter to the reporter nucleic acid molecule, followed by a second amplification reaction to add a second sequencing adapter to the reporter nucleic acid molecule.

As noted above, one or more sequencing adapters may be added to the reporter nucleic acid molecule. By this is meant one or two sequencing adapters—since sequencing adapters are added to the ends of a DNA molecule, the maximum number of sequencing adapters which can be added to a single DNA molecule (e.g. reporter nucleic acid) is two. Thus a single sequencing adapter may be added to one end of a reporter nucleic acid molecule, or two sequencing adapters may be added to a reporter nucleic acid molecule, one to each end. In a particular embodiment the IIlumina P5 and P7 adapters are used, i.e. the P5 adapter is added to one end of the reporter nucleic acid molecule and the P7 adapter is added to the other end. The sequence of the P5 adapter is set forth in SEQ ID NO: 1 (AAT GAT ACG GCG ACC ACC GA) and the sequence of the P7 adapter is set forth in SEQ ID NO: 2 (CAA GCA GAA GAC GGC ATA CGA GAT).

Thus in a particular embodiment of the invention, the reporter nucleic acid molecules are subjected to at least a first (i.e. at least one) PCR amplification, in order to add at least a first (i.e. to add at least one) sequencing adapter to the reporter nucleic acid molecule. As noted above, a reporter nucleic acid molecule is produced during the detection reaction, in response to the presence of the target analyte to which the reporter nucleic acid molecule corresponds (i.e. the analyte whose presence is indicated by generation of the reporter nucleic acid molecule). As further noted above, the reporter nucleic acid molecule is preferably amplified in order to enable or improve its detection.

This amplification may thus be combined with addition of one or more sequencing adapters to the reporter nucleic acid molecule. This may be achieved by amplification of the reporter nucleic acid molecule using a primer pair comprising at least one sequencing adapter. In this instance, at least one primer in the primer pair comprises a sequencing adapter upstream of the sequence which binds the reporter nucleic acid molecule. Thus the sequencing adapter is generally located at the 5′ end of any primer within which it is contained.

In a particular embodiment, an amplification step is performed using a primer pair comprising one primer which includes a sequencing adapter, such that a single sequencing adapter is added to one end of the reporter nucleic acid molecule.

In another embodiment, an amplification step is performed using a primer pair in which both primers comprise a sequencing adapter, such that a sequencing adapter is added to each end of the reporter nucleic acid molecule in a single amplification step.

In another embodiment, two separate amplification reactions are performed to add a sequencing adaptor to each end of the reporter nucleic acid molecule, wherein each amplification step adds a different sequencing adaptor to a different end of the molecule.

In another embodiment, an initial amplification step is performed using primers which do not comprise sequencing adapters. The amplified reporter nucleic acid molecules are then subjected to one or more further amplification reactions to add sequencing adapters to each end of the molecule, as described above.

As detailed above, each reporter nucleic acid molecule generated during the detection assay may comprise a barcode sequence which corresponds to a particular analyte. Thus reporter nucleic acid molecules with different sequences are generated in response to the presence of different analytes in the sample. Nonetheless, for ease of multiplexing it is preferred that all reporter nucleic acid molecules generated in the detection assay share common primer binding sites, such that the same primer pair can be used for amplification of all different reporter nucleic acid molecules.

If the reporter nucleic acid molecule is subjected to a first PCR amplification in which only a single sequencing adapter is added to the molecule, the amplified reporter nucleic acid molecule (that is to say, the product of the first PCR amplification) may be subjected to a second PCR amplification to add a second sequencing adapter. Thus in this embodiment a first PCR amplification is performed using a primer pair in which one primer comprises a sequencing adapter, thus adding a first sequencing adapter to one end of the reporter nucleic acid molecule. The second PCR amplification is then performed using a different primer pair. The second primer pair comprises one primer which comprises a second sequencing adapter. The second sequencing adapter is different to the first sequencing adapter, i.e. it has a different sequence. The primer comprising the second sequencing adapter binds the reporter nucleic acid molecule at the opposite end to the end comprising the first sequencing adapter, such that the second sequencing adapter is added to the reporter nucleic acid molecule at the opposite end to the first sequencing adapter.

As necessary for amplification of the product of the first PCR amplification, the second primer of the second primer pair may comprise the sequence of the first sequencing adapter, in order that it can bind to the end of the reporter nucleic acid molecule to which the first sequencing adapter was added during the first PCR amplification. In a particular embodiment, the primer comprising the first sequencing adapter used in the first PCR amplification to add the first sequencing adapter to the reporter nucleic acid molecule, is also used in the second PCR amplification. That is to say, the same primer (comprising the first sequencing adapter) may be used in both the first and second PCR amplifications.

In embodiments where two sequential PCR amplifications are performed in order to add sequencing adapters to both ends of the reporter nucleic acid molecule, the products of the first PCR may be purified before they are subjected to the second PCR. Standard methods for purification of PCR products are known in the art.

As noted above, the IIlumina P5 and P7 sequencing adapters are a preferred pair of sequencing adapters for use in the present invention. In a particular embodiment, the P5 sequencing adapter is added to the reporter nucleic acid molecule in the first PCR amplification and the P7 sequencing adapter is added to the reporter nucleic acid molecule in the second PCR amplification. In another embodiment, the P7 sequencing adapter is added to the reporter nucleic acid molecule in the first PCR amplification and the P5 sequencing adapter is added to the reporter nucleic acid molecule in the second PCR amplification.

It is preferred that at least one of the one or two PCR amplifications performed to add sequencing adapters to the reporter nucleic acid molecule is run to saturation. As is well known in the art, the amount of product of a PCR amplification relative to cycle number adopts the shape of an “S”. After a slow initial increase in amplicon concentration, a phase of exponential amplification is reached, during which the amount of product (approximately) doubles with each amplification cycle. Following the exponential phase a linear phase is reached, in which the amount of product increases in a linear, rather than exponential, fashion. Finally, a plateau is reached, in which the amount of product has reached its maximum possible level, given the reaction set-up and the concentration of components used, etc.

In the present invention, a saturated PCR may be broadly considered to be any PCR which has moved beyond the exponential phase, i.e. a PCR in linear phase or that has plateaued. In a particular embodiment, “saturation” as used herein means that the reaction is run until the maximum possible product has been obtained, such that even if more amplification cycles are performed no more product is created (i.e. that the reaction is run until the amount of product plateaus). Saturation may be reached upon depletion of a reaction component, e.g. upon primer depletion or dNTP depletion. Depletion of a reaction component results in the reaction slowing and then entering a plateau. Less commonly, saturation may be reached upon polymerase exhaustion (i.e. if the polymerase loses its activity). Saturation may also be reached if the concentration of amplicon reaches such a high level that the concentration of DNA polymerase is not sufficient to maintain exponential amplification, i.e. if there are more amplicon molecules than polymerase molecules. In this instance, so long as ample primers and dNTPs remain in the reaction mix, the amplification enters and remains in linear phase.

In a particular embodiment, two PCR amplifications are performed to add sequencing adapters to the reporter nucleic acid molecule, and both of these reactions are run to saturation. In another embodiment, only the first of the two PCR amplifications is run to saturation. Alternatively, only the second of the two PCR amplifications is run to saturation. It is particularly preferred that only the first of the two PCR amplifications is run to saturation.

A PCR amplification may be run to saturation simply by running it for a large number of cycles, such that saturation can be assumed. For instance, a PCR amplification run for at least 25, 30, 35 or more amplification cycles can be assumed to have reached saturation by the end point, in that the exponential amplification phase will have ended by that stage. Alternatively, saturation can be measured by quantitative PCR (qPCR). For instance, TaqMan PCR could be performed using a probe which binds a common sequence across all reporter nucleic acid molecules, or qPCR could be performed using a dye which changes colour upon binding to double-stranded DNA, such as SYBR Green. The reaction can thus be followed and the minimum number of amplification cycles required to reach saturation determined. Either way, given that further processing of the amplified reporter nucleic acid molecules is required (up to and including sequencing), it would be necessary to perform any such experimental qPCR to identify the point of saturation in a separate aliquot to that used experimentally to generate reporter nucleic acid molecules for sequencing, since TaqMan probes or intercalating dyes are likely to interfere with the further steps of the method.

As detailed above, separate multiplex reactions are performed for each aliquot of the sample of interest. Each aliquot is used for detection of analytes present at different levels in the sample. Reporter nucleic acid molecules will be initially generated in amounts corresponding to the amounts of each analyte in the sample. Thus for analytes present at high concentration, a high concentration of reporter nucleic acid molecule can be expected to be generated; for analytes present at low concentration, a low concentration of reporter nucleic acid molecule can be expected. It can be expected that the amount of reporter nucleic acid molecule generated will be proportionate to the amount of corresponding analyte present in the sample, e.g. for a first analyte present in the sample at ten times the concentration of a second analyte, it can be expected that ten times as much reporter nucleic acid molecule will be generated for the first analyte as for the second. Thus a much greater amount of reporter nucleic acid molecules will be generated in an aliquot used for detection of analytes expected to be present in the sample at high concentration than in an aliquot used for detection of analytes expected to be present in the sample at low concentration.

If this difference in reporter nucleic acid amount were carried through to the analysis step in which the reporter nucleic acid molecules are identified (e.g. the sequencing step), the reporter nucleic acid molecules present in the highest amounts could “drown out” the signal from reporter nucleic acid molecules present in low amounts, resulting in poor detection of the analytes present in the sample in low amounts.

Amplification of the reporter nucleic acid molecules from each multiplex reaction in a PCR run to saturation means that these differences in reporter nucleic acid concentration between aliquots will be removed. Once saturation has been reached essentially the same amount of reporter nucleic acid molecule will be present in each aliquot. This means that similar amounts of reporter nucleic acid molecule are present for each analyte present in the sample, which in turn means that all reporter nucleic acid molecules (and thus their corresponding analytes) should be detected when the reporter nucleic acid molecules are analysed.

As noted above, the multiplex detection assays used in the present method are performed on multiple separate aliquots of the sample of interest. The products of the multiplex detection assays are then used to identify which of the target analytes are present in the sample. As detailed above, this may be achieved using reporter nucleic acid molecules which correspond to different analytes, and which are analysed, e.g. by sequencing, to determine which reporter nucleic acid molecules are present (and thus which analytes are present in the sample). It is possible for each multiplex reaction performed for each sample aliquot to be analysed separately. However, in a preferred embodiment of the invention, the reaction products (i.e. the products of the multiplex detection assay) from each aliquot are pooled (that is to say mixed together). In other words, in such a pooling step, the separate “abundance blocks” can be seen to be pooled. This enables more efficient analysis of the reaction products by enabling a single analysis reaction (e.g. sequencing reaction) for all the aliquots from the sample.

If the products of the multiplex detection assay are reporter nucleic acid molecules, it is preferred that the reporter nucleic acid molecules are first amplified (e.g. by PCR), and the amplification products pooled. Optionally, a further amplification step may take place in the pool.

It is particularly preferred that the reporter nucleic acid molecules generated by each separate multiplex detection assay are subjected to a separate first PCR amplification, as described above, in which a first sequencing adapter is added to the nucleic acid molecule, the products of which are pooled. In other words, in each separate aliquot, the detection assay is performed and reporter nucleic acid molecules generated, and the reporter nucleic acid molecules subjected to a first PCR reaction which both amplifies the reporter nucleic acid molecules and adds a first sequencing adapter to one end of them. The products of this first amplification reaction are pooled. If desired, the products of each separate first PCR reaction may be purified before pooling. Alternatively, the products of the separate first PCR reactions may be pooled, and all PCR products in the pool then purified together. However, there is no requirement that the products of the first PCR amplification are purified before proceeding to the second PCR amplification.

Following pooling a second PCR amplification is performed on the pooled products of the first PCR amplification. The second PCR is used both to amplify the products of the first PCR and to add a second sequencing adapter to the reporter nucleic acid molecule, as detailed above. When the products of the first PCR are pooled, it is important that the first PCR is run to saturation, so that approximately the same amount of amplified reporter nucleic acid molecule is present in each aliquot at the time of pooling. It is not important whether the second PCR amplification, performed on the pooled products of the first PCR amplification, is also run to saturation, though it may be if desired. In a preferred embodiment, both the first and second PCR amplifications are run to saturation.

In an alternative embodiment, separate multiplex detection assays are performed for each separate aliquot. The reporter nucleic acid molecules generated in each aliquot are then subjected to a single PCR reaction, run to saturation and performed separately for each aliquot, in which sequencing adapters are added to each end of the reporter nucleic acid molecules (one sequencing adapter is added to each end of each reporter nucleic acid molecule). The products of this PCR reaction are then pooled and sequenced.

In yet another embodiment, separate multiplex detection assays are performed for each separate aliquot. The reporter nucleic acid molecules generated in each aliquot are then subjected to two PCR amplifications, both performed separately for each aliquot. The first PCR is used to add a first sequencing adapter to the reporter nucleic acid molecules, and the second PCR is used to add a second sequencing adapter to the reporter nucleic acid molecules (at the opposite end of the reporter nucleic acid molecules to the first sequencing adapter). The products of the second PCR are then pooled and sequenced. In this embodiment, it is important that at least one of the PCR amplifications is run to saturation for each aliquot. Either the first PCR or the second PCR or both PCRs may be run to saturation, so long as the same reaction(s) is/are run to saturation in each aliquot.

When pooling the amplified reporter nucleic acid molecules from each separate multiplex reaction, the same or different amounts of amplification product from each separate multiplex reaction may be added to the pool. It may be that the same amount of the amplification products obtained from each separate multiplex reaction is added to the pool. This may be achieved by adding the complete amplification reaction mixture from each multiplex reaction to the pool, or alternatively the same defined volume may be taken from each amplification reaction mixture and added to the pool. In this instance, if e.g. three aliquots are provided from the sample, a separate multiplex detection assay performed for each, and amplified reporter nucleic acid molecules from each aliquot pooled, one third of the pool will be derived from each aliquot. Equivalently, if four aliquots are provided from the sample, one quarter of pool will be derived from each aliquot.

Alternatively, different amounts of the amplification products obtained from each separate multiplex reaction may be added to the pool. By “different amounts of the amplification products” is simply meant that the amount of amplification product added to the pool is not the same across all aliquots/multiplex detection assays. Thus it may be the case that a different amount of amplification product from each multiplex detection assay is added to the pool, or alternatively the same amount of amplification product may be added from some, but not all aliquots, such that a different amount of amplification product is added from some aliquots. For instance, if three aliquots are provided from the sample, a separate multiplex detection assay performed for each, and amplified reporter nucleic acid molecules from each aliquot pooled, it may be that different amounts of amplification product are added to the pool from all three aliquots. Alternatively, the same amount of amplification product may be added to the pool from two aliquots, and a different amount from the third. Similarly, if e.g. four aliquots are provided from the sample, it may be that different amounts of amplification product are added to the pool from all four aliquots. Alternatively, the same amount of amplification product may be added to the pool from three aliquots, and a different amount from the fourth aliquot. If the same amount of amplification product is added to the pool from two aliquots, it may be that different amounts of amplification product are added to the pool from each of the other two aliquots, or it may be that a first same amount of amplification product is added to the pool from two aliquots, and a second same amount, which is different to the first same amount, is added to the pool from the other two aliquots.

If different amounts of amplification product are added to the pool from the various aliquots, the amounts of each aliquot added are preferably proportionate to the number of analytes detected in each respective aliquot. So for instance, if twice as many analytes are detected in a first aliquot as in a second aliquot, twice as much of the first aliquot is added to the pool as of the second aliquot. This may be seen as adding the same volume of amplification product to the pool for every analyte detected in the sample, across the aliquots. For instance, if 100 analytes are detected across three aliquots, 50 in the first aliquot, 30 in the second aliquot and 20 in the third aliquot, amounts of the three aliquots in the ration 5:3:2 would be added to the pool, such that 50% of the pool would be derived from the first aliquot, 30% from the second aliquot and 20% from the third aliquot.

The method of the first aspect of the invention may be used to analyse multiple samples in parallel. When multiple samples are analysed in parallel, the samples may be of the same type or of different types. Preferably all samples are of the same type, e.g. all plasma samples, or all saliva samples, etc. The set of analytes detected in each sample may also be the same or different. Preferably the same set of analytes is detected in each sample, and the same reporter nucleic acid molecule is used to identify each particular analyte in all samples. By analysing multiple samples in parallel is meant that the multiple samples are analysed at the same time, with each step of the method performed for each sample at essentially the same time.

When multiple samples are analysed in parallel, multiple aliquots are provided from each sample and a subset of analytes detected in each aliquot, as detailed above. Preferably, the same number of aliquots is provided from each sample, e.g. 3 aliquots may be provided from each sample, or 4 aliquots may be provided from each sample. However, this is not essential and it may be the case that different numbers of aliquots are provided from different samples, e.g. from some samples 2 aliquots may be provided, from others 3 aliquots, from others 4 aliquots and from others 5 aliquots.

As noted above, it is preferable that the same set of analytes is detected in each sample, and the same number of aliquots provided from each sample. It is further preferred that the analytes are divided between the aliquots in the same manner for each sample, such that in each corresponding sample aliquot (i.e. the aliquot from each sample having the same dilution factor) the same subset of analytes is detected.

When the method of the first aspect of the invention is used to analyse multiple samples in parallel, the reporter nucleic acid molecules are amplified as described above, and the amplification products for each particular sample may be pooled, as described, to generate a first pool. Separate first pools may thus be generated for each sample, and each first pool contains amplification products from all multiplex detection assays performed for its sample (i.e. amplification products from all aliquots provided for that sample).

In an embodiment the separate first pools, which are generated for each sample, may further be pooled, to facilitate subsequent analysis. In such an embodiment, following the first pooling step, a sample index is added to the amplification products in each first pool. A sample index is a nucleotide sequence which identifies the source sample from which an amplification product is derived. Thus a different nucleotide sequence is used as the sample index sequence for amplification products derived from each sample. When the amplification products are subsequently sequenced, the sample index will indicate which sample each individual reporter nucleic acid molecule is from. Any nucleotide sequence may be used as the sample index. Sample index sequences may be of any length but are preferably relatively short, e.g. 3-12, 4-10 or 4-8 nucleotides.

Thus a different sample index sequence is used to label the amplification products in each separate first pool. However, within each individual first pool, the sample index sequence is the same. The sample index sequence may be added to the amplification products by any suitable method, for instance the sample index may be added in an amplification reaction (e.g. by PCR) or in a ligation reaction. Notably, if the amplified reporter nucleic acid molecules are to be analysed by massively parallel DNA sequencing, and require sequencing adapters at both ends, the sample index sequence cannot be added such that it is, ultimately, located at an end of the reporter nucleic acid molecules.

As noted above, it is preferred that reporter nucleic acid molecules are subjected to a first PCR amplification, which includes the addition of a first sequencing adapter to the reporter molecules, and then pooled to make the first pool. This remains the case when multiple samples are analysed in parallel. It is preferred that, as described above, a first PCR amplification is performed separately for each aliquot of each sample, adding a first sequencing adapter to one end of the reporter nucleic acid molecules. The aliquots from each sample are separately pooled, as described above, to yield separate first pools for each sample.

Once the separate first pools are obtained, the sample index is added. As noted above, this may be achieved by amplification or ligation. However the sample index is added, it is added to the opposite end of the reporter nucleic acid molecule to the end comprising the first sequencing adapter. A ligation step may be performed to add the sample index to the end of each reporter nucleic acid molecule, but preferably addition of the sample index is achieved by amplification, generally by PCR. The sample index is added during amplification by using a primer pair comprising one primer which includes the sample index sequence, such that the sample index is incorporated into the amplification product.

Addition of the sample index may be performed in a dedicated amplification step which is performed exclusively to add the sample index to the reporter nucleic acid molecule. Thereafter a further amplification step may be performed, if necessary, to add a second sequencing adapter to the reporter nucleic acid molecule. In this instance, the second sequencing adapter is added to the reporter nucleic acid molecule at the same end at which the sample index is present. This would generally thus result in the sample index being located immediately adjacent to the second sequencing adapter, internal to the sequencing adapter in the amplified and adapter-tagged reporter nucleic acid molecule.

Preferably, however, following pooling of the products of the first PCR amplification to yield first pools, as detailed above, a second PCR amplification product is performed on the first pools (i.e. on the products of the first PCR amplification) which adds both a sample index and a second sequencing adapter to the reporter nucleic acid molecules. Thus a separate second PCR amplification is performed for each first pool, i.e. a separate second PCR is performed for each sample analysed.

In this embodiment, the second PCR amplification is performed with a primer pair which comprises one primer containing both the sample index sequence and the second sequencing adapter, such that both are added to the reporter nucleic acid molecules at the same time. The primer comprising the second sequencing adapter and the sample index sequence has the second sequencing adapter at its 5′ end. The sample index sequence is downstream of the second sequencing adapter, generally immediately downstream, such that it is adjacent to the second sequencing adapter, though adjacency is not required. The product of the second PCR amplification thus contains two sequencing adapters (one at each end) and a sample index, internal to the second sequencing adapter.

The second PCR may use a common first primer and a unique second primer, which differs across the multiple samples analysed. In other words, one primer (the same primer) is used across all samples to bind the end of the reporter nucleic acid molecule to which the first sequencing adapter was added in the first PCR amplification. A different second primer is used for each sample, in that the second primer comprises the sample index sequence which is unique to every sample.

Following the second PCR amplification, the indexed first pools generated for each sample are themselves pooled (i.e. added to each other, or mixed together) to create a second pool. The second pool is used for DNA sequencing. Thus a single DNA sequencing reaction can be performed to identify the reporter nucleic acid molecules generated for each sample. The sample index added to the reporter nucleic acid molecules allows each nucleic acid molecule to be traced to its sample, so that it can be determined which analytes are present in each sample. Prior to DNA sequencing, amplification products of the second PCR are preferably purified, to remove excess primer etc. left over from the amplification reaction. This purification step may be performed regardless of whether one or multiple samples are analysed in the method. If multiple samples are being analysed, and the products of the second PCR amplifications are pooled prior to sequencing, purification of the products of the second PCR may be performed before or after pooling. That is, the second PCR may be performed for each first pool, the products pooled to generate a second pool, and the PCR products in the second pool then purified together in a single purification reaction. Alternatively, the second PCR may be performed for each first pool, the products of each second PCR purified separately, and the purified products of the second PCR amplifications then pooled.

As noted above, each reporter nucleic acid molecule comprises at least one barcode sequence, which correlates to a particular analyte. Each particular reporter nucleic acid molecule is thus detected by detection of its barcode sequence, generally by sequencing. When the method of the first aspect of the invention is used to analyse a single sample, detection of all the reporter nucleic acid molecules generated in the multiplex detection assays requires only the detection of their barcodes. The detection of each particular barcode indicates the presence in the sample of its corresponding analyte. When the method is used to analyse multiple samples in parallel, following amplification each reporter nucleic acid molecule comprises both a barcode sequence and a sample index. In this embodiment detection of each reporter nucleic acid molecule comprises detection of both the barcode sequence and the sample index: detection of the sample index indicates which sample the reporter nucleic acid molecule is from, and detection of the barcode indicates the presence of a particular analyte in that sample. Thus reporter nucleic acid molecule detection enables identification of the analytes present in each sample analysed.

As noted above, sequencing for the present method is generally performed by massively parallel DNA sequencing. To this end, the purified products of the second PCR amplification (or an aliquot thereof) are denatured, using e.g. sodium hydroxide, to obtain single-stranded DNA molecules. The denatured (single-stranded) DNA may be diluted, if necessary, using a suitable buffer. Suitable dilution buffers are commonly provided with, or by the manufacturers of, DNA sequencing platforms. The denatured DNA is then loaded onto the solid support (e.g. bead or flow cell), by hybridisation of its sequencing adapters to the complementary sequences protruding from the support. Once the DNA is loaded onto the solid support, DNA sequencing is performed using the chosen method.

The methods described above enable the detection of each analyte within the sample. The method also allows comparison of the levels of analytes within each subset for each sample, i.e. it allows comparison of the levels of analytes within each particular sample aliquot analysed. Within each individual aliquot, the levels of each different reporter nucleic acid molecule generated are proportionate to the levels of their respective analytes (e.g. if a first analyte is present in a particular aliquot at twice the level of a second aliquot, twice as much reporter nucleic acid molecule corresponding to the first analyte will be generated as reporter nucleic acid molecule corresponding to the second analyte). This difference in levels of reporters will be detected during detection of the reporters, e.g. during sequencing, enabling comparison between the relative amounts of analytes present in a sample, but only for analytes detected in the same aliquot.

It is advantageous if the relative amounts of all analytes present in a sample can be compared (i.e. if comparison can be made between analytes detected in different aliquots). It is a further advantage if the relative amounts of analytes present in different samples can be compared. This can be achieved by including an internal control for each aliquot. The same internal control is included in each aliquot of each sample. The internal control is included in each aliquot of the sample at a different concentration, depending on the dilution factor of the aliquot. The concentration of the internal control is proportionate to the dilution factor of the aliquot. Thus, for instance, if the internal control is used at a particular given concentration in an undiluted sample aliquot, in a 1:10 diluted sample aliquot the internal control is used at a concentration one tenth of that used in the undiluted sample, and so on. This enables straightforward comparisons in relative concentrations of analytes between aliquots, while ensuring that the signal from the internal control does not overwhelm, and is not overwhelmed by, the signals from the analytes detected in the aliquots, as the internal control is present in each aliquot at a concentration appropriate for the analytes detected therein.

The internal control is, or results in the generation of, a control reporter nucleic acid molecule. By comparing the amount of each reporter nucleic acid molecule to the control reporter, the relative amounts of analytes analysed in different aliquots, and/or from different samples, can be compared. This is achievable because the relative difference between each reporter nucleic acid molecule and the control reporter is comparable.

For instance, if two different reporter nucleic acid molecules from different samples are present at the same relative level to the control reporter (e.g. 2- or 3-fold less or 2- or 3-fold more), this shows that the analytes indicated by the two reporter nucleic acid molecules are present at essentially the same concentrations in the two samples. Similarly, if the ratio of a particular reporter nucleic acid molecule to the control reporter is double that of the same reporter nucleic acid molecule from a different sample to the control reporter (e.g. if the reporter molecule is present in the first sample at double the level of the control reporter, and the reporter molecule is present in the second sample at essentially the same level as the control reporter), this shows that the analyte indicated by the particular reporter nucleic acid molecule is present in the first sample at approximately twice the level at which it is present in the second.

There are various alternatives which may be used as the internal control. Suitable controls may depend on the detection technique used. For any detection assay, the internal control may be a spiked analyte, i.e. a control analyte added to each aliquot analysed at a defined concentration. The control analyte added to the aliquot prior to the multiplex detection assay, and is detected in each aliquot in the same manner as the other analytes in the sample. In particular, detection of the control analyte may lead to the generation of a control reporter nucleic acid molecule, specific for the control analyte, as described above. If a control analyte is used, the control analyte is an analyte which cannot be present in the sample of interest. For instance, it may be an artificial analyte, or if the sample is derived from an animal (e.g. a human), the control analyte may be a biomolecule derived from a different species, which is not present in the animal of interest. In particular the control analyte may be a non-human protein. Exemplary control analytes include fluorescent proteins, such as green fluorescent protein (GFP), yellow fluorescent protein (YFP) and cyan fluorescent protein (CFP).

Another example of an internal control is a double-stranded DNA molecule having the same general structure as a reporter nucleic acid molecule generated in the multiplex detection assay. That is to say, the DNA molecule comprises a barcode sequence which identifies it as a control reporter nucleic acid molecule, and common primer binding sites, shared with all other reporter nucleic acids generated in response to analyte detection, to enable binding of the primers used in the amplification reaction(s). Notably the control DNA molecule does not include sequencing adapters or a sample index — these are added to the control DNA molecule at the same time as they are added to the reporter nucleic acid molecules generated in response to analyte detection, as described above (e.g. in PCR amplification).

A double-stranded DNA molecule used as a control in this manner is referred to herein as a detection control, since it is not only useful in benchmarking analyte concentrations (by comparing their concentrations relative to the control, as described above), but it also provides confirmation that reporter nucleic acid molecules generated during analyte detection are amplified, tagged and detected (e.g. by sequencing), as described above. If the detection control is not detected when the reporter nucleic acid molecules are analysed (e.g. sequenced), this indicates that the detection method has failed. For instance an amplification step may have failed, or the sequencing reaction may have failed. A detection control is preferably added to each aliquot prior to performing the multiplex detection assay.

In a particular embodiment of the method, a control analyte and a detection control are both added to each aliquot. In this instance, clearly, the barcode sequence for the control analyte is different to the barcode sequence for the detection control, so that the two internal controls can be individually identified.

As noted above, it is preferred that the multiplex detection assay is a multiplex proximity extension assay or a multiplex proximity ligation assay, most preferably a multiplex proximity extension assay. These are described briefly above. As noted above, both of these techniques rely on the use of pairs of proximity probes.

A proximity probe is defined herein as an entity comprising an analyte-binding domain specific for an analyte, and a nucleic acid domain. By “specific for an analyte” is meant that the analyte-binding domain specifically recognises and binds a particular target analyte, i.e. it binds its target analyte with higher affinity than it binds to other analytes or moieties. The analyte-binding domain is preferably an antibody, in particular a monoclonal antibody. Antibody fragments or derivatives of antibodies comprising the antigen-binding domain are also suitable for use as the analyte binding domain. Examples of such antibody fragments or derivatives include Fab, Fab′, F(ab′)₂ and scFv molecules.

A Fab fragment consists of the antigen-binding domain of an antibody. An individual antibody may be seen to contain two Fab fragments, each consisting of a light chain and its conjoined N-terminal section of the heavy chain. Thus a Fab fragment contains an entire light chain and the V_{H} and C_{H}1 domains of the heavy chain to which it is bound. Fab fragments may be obtained by digesting an antibody with papain.

F(ab′)₂ fragments consist of the two Fab fragments of an antibody, plus the hinge regions of the heavy domains, including the disulphide bonds linking the two heavy chains together. In other words, a F(ab′)₂ fragment can be seen as two covalently joined Fab fragments. F(ab′)₂ fragments may be obtained by digesting an antibody with pepsin. Reduction of F(ab′)₂ fragments yields two Fab′ fragments, which can be seen as Fab fragments containing an additional sulfhydryl group which can be useful for conjugation of the fragment to other molecules. ScFv molecules are synthetic constructs produced by fusing together the variable domains of the light and heavy chains of an antibody. Typically, this fusion is achieved recombinantly, by engineering the antibody gene to produce a fusion protein which comprises both the heavy and light chain variable domains.

The nucleic acid domain of a proximity probe may be a DNA domain or an RNA domain. Preferably it is a DNA domain. The nucleic acid domains of the proximity probes in each pair typically are designed to hybridise to one another, or to one or more common oligonucleotide molecules (to which the nucleic acid domains of both proximity probes of a pair may hybridise). Accordingly, the nucleic acid domains must be at least partially single-stranded. In certain embodiments the nucleic acid domains of the proximity probes are wholly single-stranded. In other embodiments, the nucleic acid domains of the proximity probes are partially single-stranded, comprising both a single-stranded part and a double-stranded part.

Proximity probes are typically provided in pairs, each specific for a target analyte. As noted above, a target analyte may be a single entity, in particular an individual protein. In this embodiment, both probes in the proximity pair bind the target analyte (e.g. protein), but at different epitopes. The epitopes are non-overlapping, so that the binding of one probe in the pair to its epitope does not interfere with or block binding of the other probe in the pair to its epitope. Alternatively, as noted above the target analyte may be a complex, e.g. a protein complex, in which case one probe in the pair binds one member of the complex and the other probe in the pair binds the other member of the complex. The probes bind the proteins within the complex at sites different to the interaction sites of the proteins (i.e. the sites in the proteins through which they interact with each other).

As noted above, proximity probes are provided in pairs, each specific for a target analyte. By this is meant that within each proximity probe pair, both probes comprise analyte-binding domains specific for the same analyte. Since the detection assay used is a multiplex assay, multiple different probe pairs are used in each detection assay, each probe pair being specific for a different analyte. That is to say, the analyte-binding domains of each different probe pair are specific for a different target analyte.

The nucleic acid domains of each proximity probe are designed dependent on the method in which the probes are to be used. A representative sample of proximity extension assay formats is shown schematically in FIG. 1 and these embodiments are described in detail below. In general, in a proximity extension assay, upon binding of a pair of proximity probes to their target analyte the nucleic acid domains of the two probes come into proximity of each other and interact (i.e. directly or indirectly hybridise to one another). The interaction between the two nucleic acid domains yields a nucleic acid duplex comprising at least one free 3′ end (i.e. at least one of the nucleic acid domains within the duplex has a 3′ end which can be extended). Addition or activation of a nucleic acid polymerase enzyme within the assay mix leads to extension of the at least one free 3′ end. Thus at least one of the nucleic acid domains within the duplex is extended, using its paired nucleic acid domain as template. The extension product obtained is a reporter nucleic acid molecule as used herein, comprising a barcode sequence which indicates the presence of the analyte bound by the proximity probe pair from which the extension product was produced.

Version 1 of FIG. 1 depicts a “conventional” proximity extension assay, wherein the nucleic acid domain (shown as an arrow) of each proximity probe is attached to the analyte-binding domain (shown as an inverted “Y”) by its 5′ end, thereby leaving two free 3′ ends. When said proximity probes bind to their respective analyte (the analyte is not shown in the figure) the nucleic acid domains of the probes, which are complementary at their 3′ ends, are able to interact by hybridisation, i.e. to form a duplex. The addition or activation of a nucleic acid polymerase enzyme in the assay mixture allows each nucleic acid domain to be extended using the nucleic acid domain of the other proximity probe as template. As detailed above, the resultant extension product is a reporter nucleic acid molecule which is detected, thereby detecting the analyte bound by the probe pair.

Version 2 of FIG. 1 depicts an alternative proximity extension assay, wherein the nucleic acid domain of the first proximity probe is attached to the analyte-binding domain by its 5′ end and the nucleic acid domain of the second proximity probe is attached to the analyte-binding domain by its 3′ end. The nucleic acid domain of the second proximity probe therefore has a free 5′ end (shown as a blunt arrow), which cannot be extended using a typical nucleic acid polymerase enzyme (which extend only 3′ ends). The 3′ end of the second proximity probe is effectively “blocked”, i.e. it is not “free” and it cannot be extended because it is conjugated to, and therefore blocked by, the analyte-binding domain. In this embodiment, when the proximity probes bind to their respective analyte-binding targets on the analyte, the nucleic acid domains of the probes, which share a region of complementarity at their 3′ ends, are able to interact by hybridisation, i.e. form a duplex. However, in contrast to version 1, only the nucleic acid domain of the first proximity probe (which has a free 3′ end) may be extended using the nucleic acid domain of the second proximity probe as a template, yielding an extension product (i.e. reporter nucleic acid molecule).

In version 3 of FIG. 1, like version 2, the nucleic acid domain of the first proximity probe is attached to the analyte-binding domain by its 5′ end and the nucleic acid domain of the second proximity probe is attached to the analyte-binding domain by its 3′ end. The nucleic acid domain of the second proximity probe therefore has a free 5′ end (shown as a blunt arrow), which cannot be extended. However, in this embodiment, the nucleic acid domains which are attached to the analyte binding domains of the respective proximity probes do not have regions of complementarity and therefore are unable to form a duplex directly. Instead, a third nucleic acid molecule is provided that has a region of homology with the nucleic acid domain of each proximity probe. This third nucleic acid molecule acts as a “molecular bridge” or a “splint” between the nucleic acid domains. This “splint” oligonucleotide bridges the gap between the nucleic acid domains, allowing them to interact with each other indirectly, i.e. each nucleic acid domain forms a duplex with the splint oligonucleotide.

Thus, when the proximity probes bind to their respective analyte-binding targets on the analyte, the nucleic acid domains of the probes each interact by hybridisation, i.e. form a duplex, with the splint oligonucleotide. It can be seen therefore that the third nucleic acid molecule or splint may be regarded as the second strand of a partially double stranded nucleic acid domain provided on one of the proximity probes. For example, one of the proximity probes may be provided with a partially double-stranded nucleic acid domain, which is attached to the analyte binding domain via the 3′ end of one strand and in which the other (non-attached) strand has a free 3′ end. Thus such a nucleic acid domain has a terminal single stranded region with a free 3′ end. In this embodiment the nucleic acid domain of the first proximity probe (which has a free 3′ end) may be extended using the “splint oligonucleotide” (or single stranded 3′ terminal region of the other nucleic acid domain) as a template. Alternatively or additionally, the free 3′ end of the splint oligonucleotide (i.e. the unattached strand, or the 3′ single-stranded region) may be extended using the nucleic acid domain of the first proximity probe as a template.

As is apparent from the above description, in one embodiment, the splint oligonucleotide may be provided as a separate component of the assay. In other words it may be added separately to the reaction mix (i.e. added separately to the proximity probes to the sample containing the analytes). Notwithstanding this, since it hybridises to a nucleic acid molecule which is part of a proximity probe, and will do so upon contact with such a nucleic acid molecule, it may nonetheless be regarded as a strand of a partially double-stranded nucleic acid domain, albeit that it is added separately. Alternatively, the splint may be pre-hybridised to one of the nucleic acid domains of the proximity probes, i.e. hybridised prior to contacting the proximity probe with the sample. In this embodiment, the splint oligonucleotide can be seen directly as part of the nucleic acid domain of the proximity probe, i.e. wherein the nucleic acid domain is a partially double-stranded nucleic acid molecule, e.g. the proximity probe may be made by linking a double-stranded nucleic acid molecule to an analyte-binding domain (preferably the nucleic acid domain is conjugated to the analyte-binding domain by a single strand) and modifying said nucleic acid molecule to generate a partially double-stranded nucleic acid domain (with a single-stranded overhang capable of hybridising to the nucleic acid domain of the other proximity probe).

Hence, the extension of the nucleic acid domain of the proximity probes as defined herein encompasses also the extension of the “splint” oligonucleotide. Advantageously, when the extension product arises from extension of the splint oligonucleotide, the resultant extended nucleic acid strand is coupled to the proximity probe pair only by the interaction between the two strands of the nucleic acid molecule (by hybridisation between the two nucleic acid strands). Hence, in these embodiments, the extension product may be dissociated from the proximity probe pair using denaturing conditions, e.g. increasing the temperature, decreasing the salt concentration etc.

Whilst the splint oligonucleotide depicted in Version 3 of FIG. 1 is shown as being complementary to the full length of the nucleic acid domain of the second proximity probe, this is merely an example and it is sufficient for the splint to be capable of forming a duplex with the ends (or near the ends) of the nucleic acid domains of the proximity probes, i.e. to form a bridge between the nucleic acid domains of the two probes.

In another embodiment, the splint oligonucleotide may be provided as the nucleic acid domain of a third proximity probe as described in WO 2007/107743, which is incorporated herein by reference, which demonstrates that this can further improve the sensitivity and specificity of proximity probe assays.

Version 4 of FIG. 1 is a modification of Version 1, wherein the nucleic acid domain of the first proximity probe comprises at its 3′ end a sequence that is not fully complementary to the nucleic acid domain of the second proximity probe. Thus, when said proximity probes bind to their respective analyte the nucleic acid domains of the probes are able to interact by hybridisation, i.e. to form a duplex, but the extreme 3′ end of the nucleic acid domain (the part of the nucleic acid molecule comprising the free 3′ hydroxyl group) of the first proximity probe is unable to hybridise to the nucleic acid domain of the second proximity probe and therefore exists as a single stranded, unhybridised, “flap”. On the addition or activation of a nucleic acid polymerase enzyme, only the nucleic acid domain of the second proximity probe may be extended using the nucleic acid domain of the first proximity probe as template.

Version 5 of FIG. 1 could be viewed as a modification of Version 3. However, in contrast to Version 3, the nucleic acid domains of both proximity probes are attached to their respective analyte-binding domains by their 5′ ends. In this embodiment the 3′ ends of the nucleic acid domains are not complementary and hence the nucleic acid domains of the proximity probes cannot interact or form a duplex directly. Instead, a third nucleic acid molecule is provided that has a region of homology with the nucleic acid domain of each proximity probe. This third nucleic acid molecule acts as a “molecular bridge” or a “splint” between the nucleic acid domains. This “splint” oligonucleotide bridges the gap between the nucleic acid domains, allowing them to interact with each other indirectly, i.e. each nucleic acid domain forms a duplex with the splint oligonucleotide. Thus, when the proximity probes bind to their respective analyte, the nucleic acid domains of the probes each interact by hybridisation, i.e. form a duplex, with the splint oligonucleotide.

In accordance with Version 3, it can be seen therefore that the third nucleic acid molecule or splint may be regarded as the second strand of a partially double stranded nucleic domain provided on one of the proximity probes. In a preferred example, one of the proximity probes may be provided with a partially double-stranded nucleic acid domain, which is attached to the analyte binding domain via the 5′ end of one strand and in which the other (non-attached) strand has a free 3′ end. Thus such a nucleic acid domain has a terminal single stranded region with at least one free 3′ end. In this embodiment the nucleic acid domain of the second proximity probe (which has a free 3′ end) may be extended using the “splint oligonucleotide” as a template. Alternatively or additionally, the free 3′ end of the splint oligonucleotide (i.e. the unattached strand, or the 3′ single-stranded region of the first proximity probe) may be extended using the nucleic acid domain of the second proximity probe as a template.

As discussed above in connection with Version 3, the splint oligonucleotide may be provided as a separate component of the assay. On the other hand, since it hybridises to a nucleic acid molecule which is part of a proximity probe, and will do so upon contact with such a nucleic acid molecule, it may be regarded as a strand of a partially double-stranded nucleic acid domain, albeit that it is added separately. Alternatively, the splint may be pre-hybridised to one of the nucleic acid domains of the proximity probes, i.e. hybridised prior to contacting the proximity probe with the sample. In this embodiment, the splint oligonucleotide can be seen directly as part of the nucleic acid domain of the proximity probe, i.e. wherein the nucleic acid domain is a partially double-stranded nucleic acid molecule, e.g. the proximity probe may be made by linking a double-stranded nucleic acid molecule to an analyte-binding domain (preferably the nucleic acid domain is conjugated to the analyte-binding domain by a single strand) and modifying said nucleic acid molecule to generate a partially double-stranded nucleic acid domain (with a single-stranded overhang capable of hybridising to the nucleic acid domain of the other proximity probe).

Hence, the extension of the nucleic acid domain of the proximity probes as defined herein encompasses also the extension of the “splint” oligonucleotide. Advantageously, when the extension product arises from extension of the splint oligonucleotide, the resultant extended nucleic acid strand is coupled to the proximity probe pair only by the interaction between the two strands of the nucleic acid molecule (by hybridisation between the two nucleic acid strands). Hence, in these embodiments, the extension product may be dissociated from the proximity probe pair using denaturing conditions, e.g. increasing the temperature, decreasing the salt concentration etc.

Whilst the splint oligonucleotide depicted in Version 5 of FIG. 1 is shown as being complementary to the full length of the nucleic acid domain of the first proximity probe, this is merely an example and it is sufficient for the splint to be capable of forming a duplex with the ends (or near the ends) of the nucleic acid domains of the proximity probes, i.e. to form a bridge between the nucleic acid domains of the proximity probes.

In another embodiment, the splint oligonucleotide may be provided as the nucleic acid domain of a third proximity probe as described in WO 2007/107743, which is incorporated herein by reference, which demonstrates that this can further improve the sensitivity and specificity of proximity probe assays.

Version 6 of FIG. 1 is the most preferred embodiment of the present invention. As depicted, both probes in a pair are conjugated to partially single-stranded nucleic acid molecules. A short nucleic acid strand is conjugated via its 5′ end to the analyte-binding domain. The short nucleic acid strands which are conjugated to the analyte-binding domains do not hybridise to each other. Rather, each short nucleic acid strand is hybridised to a longer nucleic acid strand, which has a single-stranded overhang at its 3′ end (that is to say, the 3′ end of the longer nucleic acid strand extends beyond the 5′ end of the shorter strand conjugated to the analyte-binding domain. The overhangs of the two longer nucleic acid strands hybridise to one another, forming a duplex. If the 3′ ends of the two longer nucleic acid molecules hybridise fully to one another, as shown, the duplex comprises two free 3′ ends, though the 3′ ends of the longer nucleic acid molecules may be designed as in Version 4, such that the extreme 3′ end of one of the longer nucleic acid molecules is not complementary to the other, forming a flap, meaning that the duplex contains only one free 3′ end. The two longer nucleic acid molecules which interact with one another may be seen as splint oligonucleotides, in that together they form a bridge between the two short oligonucleotides which are directly conjugated to the analyte-binding domains.

Addition or activation of a nucleic acid polymerase results in extension of the free 3′ end or ends of the splint oligonucleotides. Notably, extension of either splint oligonucleotide uses the other splint oligonucleotide as template. Thus, when one splint oligonucleotide is extended, the other “template” splint oligonucleotide is displaced from the shorter strand which is conjugated to the analyte-binding domain.

In a preferred embodiment, the short nucleic acid strand conjugated directly to the analyte-binding domain is a “universal strand”. That is to say, the same strand is conjugated directly to every proximity probe used in the multiplex detection assay. Each splint oligonucleotide therefore comprises a “universal site”, which consists of the sequence which hybridises to the universal strand, and a “unique site”, which comprises a barcode sequence unique to the probe. Such proximity probes, and methods for making them, are described in WO 2017/068116.

In all proximity detection assay techniques, it is preferred that the nucleic acid domain of each individual proximity probe comprises a unique barcode sequence, which identifies the particular probe (as described above for PEA Version 6). In this case, the reporter nucleic acid molecule (which in the context of proximity extension assays is the extension product) comprises the unique barcode sequence of each proximity probe. These two unique barcode sequences thus together form the barcode sequence of the reporter nucleic molecule. In other words, the reporter nucleic acid molecule barcode sequence is comprises a combination of two probe barcode sequences, from the proximity probes which combined to generate the reporter nucleic acid molecule. Detection of a particular reporter nucleic acid molecule is thus achieved by detecting a particular combination of two probe barcode sequences.

When a multiplex proximity extension assay is used for analyte detection, it is preferred that an additional internal control is used: an extension control. The extension control is a single probe comprising an analyte-binding domain conjugated to a nucleic acid domain which comprises a duplex comprising a free 3′ end, which can be extended. The extension control preferably has a structure essentially equivalent to the duplex formed between two experimental probes upon their binding to their target analyte, except it comprises only a single analyte-binding domain. The analyte-binding domain used in the extension control does not recognise an analyte likely to be present in the sample of interest. A suitable analyte-binding domain is a commercially available, polyclonal isotype control antibody, such as goat IgG, mouse IgG, rabbit IgG, etc.

FIG. 2 shows examples of extension controls which can be used in the present invention. Parts A-F correspond to extension controls which can be used in PEA assay Versions 1-6 of FIG. 1, respectively. The extension control is used to confirm that the extension step takes place as intended. Extension of the extension control yields a reporter nucleic acid molecule which comprises a unique barcode, such that it may be identified as the extension control reporter nucleic acid molecule. When a multiplex PEA is used in the method of the first aspect of the invention, it is preferred that a control analyte, an extension control and a detection control are all used in the assay (i.e. are added to each aliquot). In other embodiments only two of the internal controls are used, e.g. a control analyte and an extension control, a control analyte and a detection control, or an extension control and a detection control.

As detailed above, in a proximity extension assay, the reporter nucleic acid molecule is generated by extension of the nucleic acid domain of one or both proximity probes, using the nucleic acid domain of the other proximity probe as template. In a preferred embodiment, an extension reaction is performed in the context of a PCR amplification, or in other words a single reaction, including a PCR amplification, is performed to achieve both extension of the proximity probe nucleic acid domains, thus generating the reporter nucleic acid molecule, and amplification of the generated reporter nucleic acid molecule, including the addition of a first sequence adapter to the reporter molecule. In this embodiment, rather than beginning with a denaturation step (as is normally the case in PCR), the reaction begins with an extension step, during which the reporter nucleic acid molecule is generated. Thereafter, a standard PCR is performed to amplify the reporter nucleic acid molecule, beginning with denaturation of the reporter molecule. As detailed above, the PCR is performed using common primers which bind to common sequences at the ends of the reporter nucleic acid molecule, and one of the primers comprises a sequencing adapter. Alternatively, the PCR may be performed using primers which both comprise sequencing adapters, in order to add a sequencing adapter to each end of the reporter nucleic acid molecule in one go, as detailed above.

It may be that it is desired to detect more analytes in a sample than are available different reporter nucleic acid barcode sequences. In this case, multiple panels (i.e. at least 2 panels) of proximity probe pairs may be used. Each panel comprises a different set of proximity probe pairs. That is to say, the proximity probe pairs in each panel bind a different set of analytes. In general, the proximity probe pairs in each panel bind a completely different set of analytes, i.e. there is no overlap in analytes bound by the proximity probe pairs in different panels. It can thus be seen that each panel of proximity probes is for the detection of a different group of analytes.

As noted above, each panel of proximity probes comprises a different set of proximity probe pairs. Within each individual panel, every probe comprises a different nucleic acid domain (i.e. every probe comprises a nucleic acid domain with a different sequence). Thus every probe pair comprises a different pair of nucleic acid domains, and so a unique reporter nucleic acid molecule is generated for each probe pair within a panel. However, the same nucleic acid domains (and generally the same nucleic acid domain pairings) are used in the probe pairs in each different panel. That is to say, in different panels the probe pairs comprise the same pairs of nucleic acid domains. This means that the same reporter nucleic acid molecules are generated in every panel. However, because the reporter nucleic acid molecules are generated by each panel using different probe pairs, the same reporter nucleic acid molecule denotes the presence of a different analyte in every panel of probes.

Since the same reporter nucleic acid molecules are generated by each panel of probes, separate sample aliquots must be provided for the multiplex detection assay using each panel of probes. That is to say, multiplex detection assays are performed using each panel of probes, and multiplex detection assays using different panels of probes are performed in different aliquots of the sample. As detailed above for a single probe panel, for each panel of probes, multiple sample aliquots are provided at different dilution factors, and a different subset of analytes from each panel is detected in each aliquot. As detailed above, the subset of analytes detected in each aliquot is determined based on their predicted concentration in the sample.

The reporter nucleic acid molecules generated using each separate probe panel are processed (i.e. amplified and possible tagged with sequencing adapters, etc.) and detected as detailed above. In a particular embodiment, the reporter nucleic acid molecules are amplified by PCR, sequencing adapters are added to both ends of the reporter nucleic acid molecules, and a sample index is added to each reporter nucleic acid molecules, as detailed above. In this embodiment, it is preferred that, as described above, a separate first PCR is performed in each aliquot, amplifying the reporter nucleic acid molecules and adding a first sequencing adapter to one end of the reporter nucleic acid molecules. Thereafter, the amplified reporter nucleic acid molecules from each sample, generated with a particular probe panel, are pooled, as described above, generating multiple separate first pools. Each separate first pool comprises the products of the first PCR amplification of all aliquots of a particular sample assayed with a particular probe panel.

A second PCR amplification is then performed in each separate first pool, in which a second sequencing adapter and a sample index is added to each reporter nucleic acid molecule. Following the second PCR, the PCR products generated from different samples but using the same probe panel are themselves pooled into a second pool, known as a panel pool. It may be that the entirety of each first pool is combined to yield the panel pool, or alternatively only a portion of each first pool may be combined. Each panel pool thus comprises the reporter nucleic acid molecules generated from all assayed samples with a particular probe panel.

The amplified reporter nucleic acid molecules comprising sequencing adapters and sample index are then sequenced, as described above. Each panel pool is sequenced separately. This is because, as noted above, the same reporter nucleic acid molecules are generated for each probe panel, but in each probe panel denote a different analyte. It is impossible to distinguish, at a sequence level, between identical reporter nucleic acid molecules generated using different probe panels and thus denoting different analytes. Accordingly, in this embodiment it is essential that each panel pool is sequenced separately.

In another embodiment of the method, a panel index sequence is added to the reporter nucleic acid molecule during one of the PCR amplifications. The same panel index sequence would be used to identify all reporter nucleic acid molecules (across all samples) generated using a particular proximity probe panel. The combination of panel index and sample index enable identification of exactly which analytes are present in each sample across all panels of probes used in the detection assay. Accordingly, once both the panel index and sample index have been added to each reporter nucleic acid molecule, all PCR products generated in the detection assay, across all samples and probe panels, can be pooled and sequenced together.

Alternatively, different sample indexes may be used to label reporter nucleic acid molecules generated using each probe panel. Different selections of sample index sequences are used for each different sample, such that every sample index used is specific to a particular sample. However, for any given sample, reporter nucleic acid molecules generated using each different probe panel are labelled with a different sample index. In this embodiment the sample index thus serves a dual function, identifying both the sample and probe panel for each reporter nucleic acid molecule. The particular sample index present in a reporter nucleic acid molecule thus links that reporter to a particular probe panel, and the combination of the sample index and the barcode sequence of the reporter nucleic acid molecule serves to identify the analyte which has led to the generation of the reporter nucleic acid molecule in question.

In a further embodiment of the method, as detailed above, in each probe panel the same nucleic acid domains are used in the probes. However, the nucleic acid domains are paired differently in each panel, such that each panel generates different reporter nucleic acid molecules. As noted above, the nucleic acid domain of each probe comprises a unique barcode sequence. By pairing the nucleic acid domains differently in each panel, different combinations of barcode sequences are paired in the reporter nucleic acid molecules generated in the detection assay, meaning that different reporter nucleic acid molecules are generated for every panel. This method has the advantage that different reporter nucleic acid molecules are generated by each probe panel, and thus can be distinguished at a sequence level without any need for a panel index sequence. In this embodiment, all PCR products from each sample are pooled as detailed above, and sample index added, and all indexed PCR products, from all samples and probe panels, then combined in a single pool which is sequenced.

As noted, the advantage of this embodiment is that all reporter nucleic acid molecules from all samples and panels can be pooled and sequenced together without the need for a panel index to identify which reporter nucleic acid molecules are derived from each panel. However, an advantage of using probe pairs with the same pairs of nucleic acid domains for each panel, such that the same reporter nucleic acid molecules are generated by each panel, is that any nucleic acid molecules generated as a result of hybridisation of two unpaired nucleic acid molecules can be identified as non-specific background. If different reporter nucleic acid molecules are generated using each probe panel, it is no longer possible to determine exactly which nucleic acid molecules generated are background.

As noted above, in a second aspect the present invention provides a method of detecting an analyte in a sample, wherein the analyte is detected by detecting a reporter nucleic acid molecule specific for the analyte, said method comprising performing a PCR reaction to generate a PCR product of the reporter nucleic acid molecule and detecting said PCR product;

wherein an internal control is provided for the PCR reaction, and said internal control is:

(i) a separate component which is present in a pre-determined amount, and which is, or comprises, or leads to the generation of, a control nucleic acid molecule which is amplified by the same primers as the reporter nucleic acid molecules; or

(ii) a unique molecular identifier (UMI) sequence present in each reporter nucleic acid molecule, which is unique to each molecule.

All details of this second aspect of the invention may be the same as in the first aspect (e.g. the analyte, the sample, the reporter nucleic acid molecule and the technique used to generate it, the detection of the reporter nucleic acid molecule, etc.).

In this second aspect, the internal control is a component or sequence present in the PCR performed to generate a PCR product of the reporter nucleic acid molecule. As noted above, the internal control may be a separate component which is present in a pre-determined amount, and which is, or comprises, or leads to the generation of, a control nucleic acid molecule which is amplified by the same primers as the reporter nucleic acid molecules.

When the internal control is a separate component which is present in the reaction in a pre-determined amount, the internal control may in particular be a control analyte, an extension control or a detection control, as described above. As detailed above, a control analyte is an analyte which is added to the sample and is detected by detecting a control reporter nucleic acid molecule specific for the control analyte.

In the method of the second aspect of the invention, the analyte is preferably detected using proximity probes, e.g. in a PEA or PLA as detailed above, most preferably a PEA. Thus, when a control analyte is used as an internal control, proximity probes for detecting the control analyte must be included. Binding of the control-specific proximity probes to the control analyte results in generation of the control reporter nucleic acid molecule.

As mentioned, an extension control may be used. As detailed above, an extension control is a single control probe, from which a control reporter nucleic acid molecule is generated during the extension stage of a PEA.

Generally speaking, the internal control may be a molecule, or molecules, which is/are added to the sample and lead(s) to the generation of a control reporter nucleic acid molecule which is then amplified in the PCR reaction.

As also mentioned, a detection control may be used. As detailed above, a detection control is a control reporter nucleic acid molecule which is added to the sample and amplified in the PCR reaction. The detection control is a double-stranded DNA molecule having the same general structure as a reporter nucleic acid molecule generated in response to the presence of an analyte. As for the first aspect of the invention, it is preferred that a control analyte, an extension control and a detection control are all used in the method. In particular embodiments, two types of internal control may be used, options for which are set out above.

As detailed above, all of a control analyte, an extension control and a detection control lead to the generation of, or are, control reporter nucleic acid molecules. In a particular embodiment of the invention, the control reporter nucleic acid molecule has a sequence which is the reverse sequence of a reporter nucleic acid molecule generated in response to detection of an analyte. Notably the control reporter nucleic acid molecule has the reverse sequence of a reporter nucleic acid molecule generated in response to detection of an analyte, and not the reverse complement sequence. Since the control reporter nucleic acid molecule has merely the reverse sequence of a reporter nucleic acid molecule generated in response to detection of an analyte, the control reporter nucleic acid molecule cannot hybridise to the reporter nucleic acid molecule in question. This allows maintenance of a maximum level of similarity between the control reporter nucleic acid molecule and the reverse sequence reporter nucleic acid molecule generated in response to detection of an analyte, which is advantageous in PCR amplification, while avoiding unwanted hybridisation interactions between the control reporter nucleic acid molecule and reporter nucleic acid molecule generated in response to detection of an analyte. A control reporter nucleic acid molecule which has a sequence which is the reverse sequence of a reporter nucleic acid molecule generated in response to detection of an analyte is preferably also used in the method of the first aspect of the invention.

As mentioned above, it is preferred that the method of this aspect of the invention uses a control analyte, an extension control and a detection control as internal controls. In order for these three controls to function together, it is apparent that the control reporter nucleic acid molecules generated/provided by the controls must be distinguishable from one another, i.e. must all have different sequences. It is preferred that each control reporter nucleic acid molecule used in the methods of the invention has a sequence which is a reverse sequence of a reporter nucleic acid molecule generated in response to detection of an analyte. In this case, clearly each control reporter nucleic acid molecule has the reverse sequence of a different reporter nucleic acid molecule generated in response to detection of an analyte.

Instead of a separate component of the amplification reaction, the internal control may alternatively be a unique molecular identifier (UMI) sequence present in each reporter nucleic acid molecule, which is unique to each molecule. By this is meant that each individual reporter nucleic acid molecule generated during analyte detection comprises a UMI sequence. More particularly, it will be understood that each individual reporter nucleic acid molecule will have a different UMI. The UMI will be additional to any sequence, e.g. barcode, which is present in the reporter nucleic acid molecule as the means for detecting or identifying an analyte. As detailed above, it is preferred that the analyte is detected according to the method of the second aspect of the invention by proximity extension assay. PEA, including probes which may be used for it, are described above. As detailed, an analyte is detected using a pair of proximity probes, which each bind the analyte. Both probes in a pair comprise a nucleic acid domain, comprising a barcode sequence which is specific for the analyte recognised by the probes.

Ordinarily when a PEA is performed multiple identical probe pairs for each analyte to be detected are applied to the sample. By “identical” probe pairs is meant that the multiple probe pairs all comprise the same pair of analyte-binding molecules, and the same pair of nucleic acid domains, such that every identical probe pair which binds a target analyte causes the generation of an identical reporter nucleic acid molecule, which is indicative of the presence of that analyte in the sample.

When UMI sequences are utilised as the internal control, the probes used to detect each particular analyte are not identical. While a particular pair of analyte-binding molecules is used, each individual probe, or at least each individual probe comprising a particular one of the two analyte-binding molecules in the pair, comprises a different, unique nucleic acid domain. Each nucleic acid domain is rendered unique by the presence of a UMI sequence within it. This means that each specific pair of probes which binds to a particular analyte molecule leads to the generation of a unique reporter nucleic acid molecule. A unique reporter nucleic acid molecule is generated for every individual analyte molecule bound by a proximity probe pair. This allows for absolute quantification of the amount of the analyte present in the sample, since the precise number of analyte molecules detected can be counted based on the number of unique reporter nucleic acid molecules generated for that particular analyte.

As well as allowing quantitation, by allowing to count backwards to the number of reporter nucleic acid molecules that are generated in the detection assay, UMIs may be advantageous as they increase the resolution of the readout. A UMI allows it to be seen how many times a reporter nucleic acid molecule (e.g. an extension product of a PEA) has been amplified. Accordingly, differences in the levels of UMIs for reporter molecules for the same analyte can be detected. For example, each individual reporter nucleic acid molecule for the same analyte may have the same barcode sequence, but a different UMI. By detecting these differences in the levels of different UMIs, any possible bias in the PCR reaction can be detected and accounted for.

The improved resolution may also be useful or beneficial in the context of control nucleic acid molecules. Accordingly, UMIs may alternatively or additionally be included in control nucleic acid molecules. Thus a UMI may be included, in the sense of added to, each individual control reporter nucleic acid molecule, e.g. a detection control molecule as discussed above (it will be understood that each individual control nucleic acid molecule will have a different UMI). Alternatively, for different IC control formats, e.g. extension controls or control analytes, UMIs can be included as appropriate, such that they are included in the control reporter nucleic acid molecule that is generated. For example, a UMI may be included within the nucleic acid sequence in the nucleic acid domain of the extension control that acts as the template for the extension reaction, or in the sequence in the part of the domain that acts as the primer for the extension reaction. Analogously, in the case of a control analyte, the UMI may be included in one or both of the nucleic acid domains of the proximity probes which are used to detect the control analyte, in such a way that it becomes incorporated into the control reporter nucleic acid.

If UMIs are included in control nucleic acids they can be used to increase resolution in normalisation. For example, they allow any PCR bias to be accounted for as discussed above. This may allow a very stringent value to be used for normalisation. Thus, UMIs may be used as a tool to improve or to secure the quality of the data.

In one exemplary embodiment, a control reporter nucleic acid molecule comprises a sequence which is the reverse sequence of a reporter nucleic acid molecule generated in response to detection of an analyte, and a UMI.

UMI sequences may also be used in the proximity probes used in the method of the first aspect of the invention.

The method of the second aspect of the invention may be applied to the detection of multiple analytes in the same sample (indeed this is preferred). As detailed above, multiple analytes may be detected in a multiplex detection assay. Each different analyte is detected based on the detection of a reporter nucleic acid molecule specific for that analyte. As detailed above, although the reporter nucleic acids for each different analyte have a unique barcode sequence, providing the specificity for the analyte, it is preferred that all reporter nucleic acids comprise common primer binding sites, such that the same primers can be used to amplify all reporter nucleic acid molecules in a single PCR. The PCR amplification of the reporter nucleic acid molecules may include the addition of at least one (i.e. one or two) sequencing adapters to the ends of the reporter nucleic acid molecule, as detailed above.

As detailed above, when multiple analytes in the same sample are detected, different subsets of the analytes may be detected in different aliquots of the sample, based on the predicted abundance of the analytes in the sample, as detailed above. In this embodiment, separate PCRs are performed for each aliquot. The PCR products may subsequently be pooled, as detailed above.

The method of the second aspect of the invention may also be used to detect an analyte, or multiple analytes, in multiple samples. In this embodiment, a separate PCR is performed to amplify the reporter nucleic acid molecules generated from each sample. Where different subsets of analytes are detected in separate aliquots for each sample, a separate PCR is performed for each aliquot of each sample. The same primers are used to amplify the reporter nucleic acid molecules generated for all analytes in all samples.

Where multiple separate PCR amplifications are performed, for multiple different samples and/or for multiple different sample aliquots, when the internal control is a separate component which is present in the PCR mix, that component is present in each aliquot at a concentration proportionate to the dilution factor of the aliquot, as described above. While the concentration of the internal control varies between aliquots of different dilution factors, the concentration of the internal control is the same in aliquots of the same dilution factor from different samples (the same is true in the first aspect of the invention). This enables comparison of the relative amounts of each analyte present in each sample/aliquot, as detailed above.

It is preferred that in the method of the second aspect of the invention the PCR reaction is run to saturation. Saturation of PCR reactions is described above. This is particularly advantageous when the method is used to detect multiple analytes of varying levels of abundance in the one or more samples, with detection assays being performed on multiple aliquots of each sample, a subset of the analytes being detected in each aliquot, as described above. The combination of running the PCR to saturation, and using a separate component of the PCR mix as an internal control, is a particularly preferred embodiment of the invention. As detailed above, running a PCR to saturation allows the differences in concentration of reporter nucleic acid molecules between different sample aliquots to be removed: once saturation is reached, essentially the same overall concentration of reporter nucleic acid molecules will be present in each reaction. The inclusion of an internal control in the reaction ensures that the ability to compare the relative levels of the analytes detected in different aliquots, or in different samples, is retained.

As noted above, it is particularly preferred that in the second aspect of the invention the one or more analytes are detected using analyte-specific probes. When such probes are used for analyte detection, the internal control (if a separate component of the PCR mixture) is generally added to the sample before the probes are added to the sample, or at the same time as the probes are added to the sample. Alternatively, as mentioned above, the internal control may constitute UMI sequences present within each probe.

Preferably, the one or more analytes are detected by a proximity assay (e.g. PEA or PLA, particularly PEA) which generates a reporter nucleic acid molecule specific for each analyte. In this embodiment, it is preferred that at least an extension control is included. As noted above, it is most preferred that a control analyte, an extension control and a detection control are all included.

In a preferred embodiment of the second aspect of the invention, the method is for detecting multiple analytes in a sample, wherein the analytes have varying levels of abundance in the sample and the method comprises:

(i) providing multiple aliquots from the sample; and

(ii) in each aliquot detecting a subset of the analytes, by performing a separate multiplex assay for each aliquot; wherein the analytes in each subset are selected based on their predicted abundance in the sample, and

wherein each aliquot comprises at least one internal control.

All parts of this embodiment may be as defined above in respect of the first aspect of the invention. The internal control may be any internal control as defined above.

As noted above, where subsets of analytes are detected in multiple aliquots, the aliquots having different dilution factors relative to the original sample, a different amount of the internal control is added. The amount of internal control added to each aliquot is determined by the predicted abundance of the subset of analytes detected in that aliquot. As detailed above, this means in practice that the amounts of internal control used in each aliquot are proportionate to the dilution factors of the aliquots.

It is preferred that the reporter nucleic acid molecules generated in the method of the second aspect of the invention (or more precisely, the PCR products resulting from the amplification of the reporter nucleic acid molecules) are detected by DNA sequencing. Most preferably, massively parallel DNA sequencing is used, as described above.

The third aspect of the invention provides a method of detecting an analyte in a sample, wherein the analyte is detected by detecting a reporter nucleic acid molecule for the analyte, said method comprising performing a PCR reaction to generate a PCR product of the reporter nucleic acid molecule and detecting said PCR product, wherein an internal control is included in the PCR reaction and said internal control is present in a pre-determined amount and is, or comprises, or leads to the generation of, a control nucleic acid molecule wherein the control nucleic acid molecule comprises a sequence which is the reverse sequence of the reporter nucleic acid molecule.

All features of the third aspect of the invention may be as described in relation to the first and/or second aspects of the invention.

The invention may be further understood by reference to the non-limiting examples below, and the figures.

### BRIEF DESCRIPTION OF FIGURES

FIG. 1 shows a schematic representation of six different versions of proximity extension assays, described in detail above. The inverted ‘Y’ shapes represent antibodies, as an exemplary proximity probe analyte-binding domain.

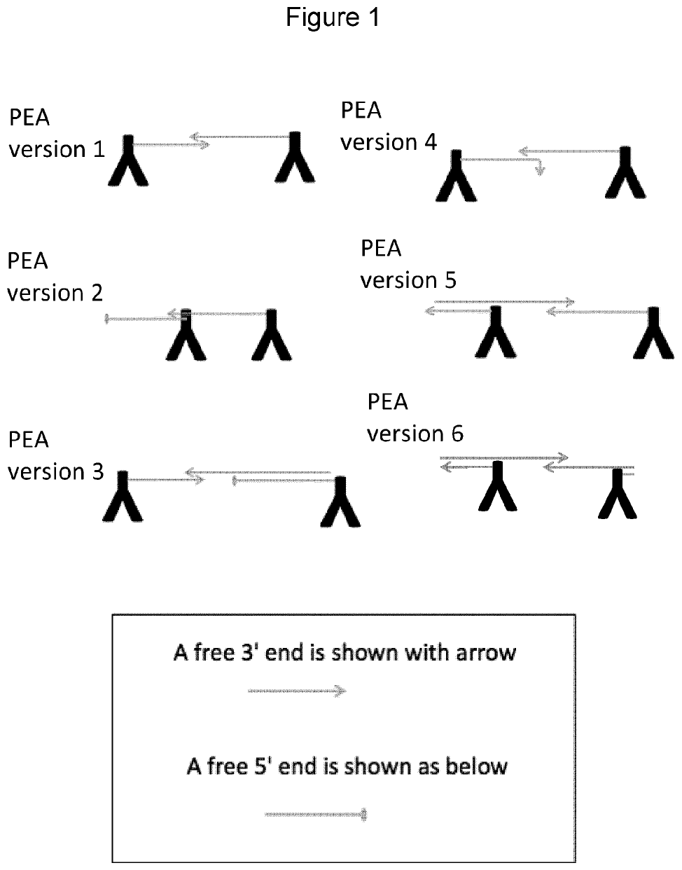

FIG. 2 shows a schematic representation of examples of extension controls which may be used in proximity extension assays. Parts A-F show suitable extension controls for use in versions 1-6 of FIG. 1, respectively. In parts B-E, different possible extension controls for use in versions 2-5 of FIG. 1, respectively, are shown in options (i) and (ii). The legend for FIG. 1 also applies to FIG. 2.

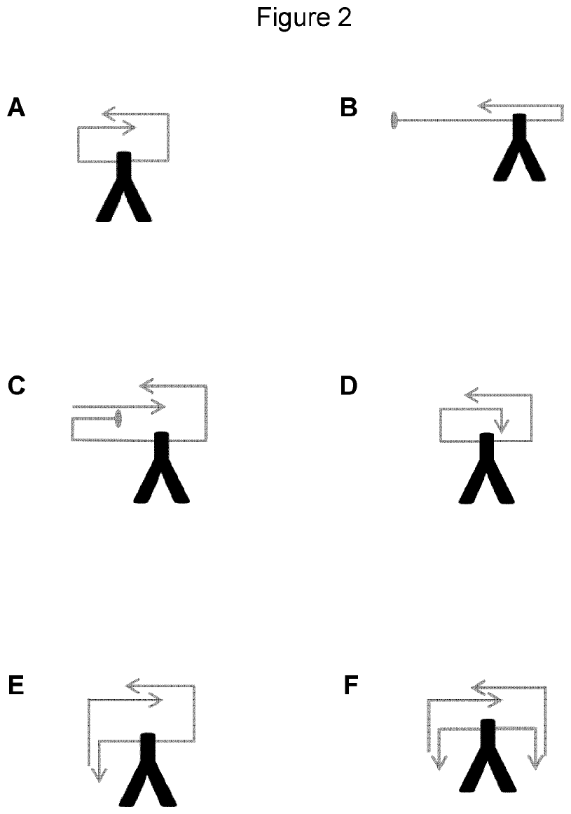

FIG. 3 shows the resulting counts (correctly paired barcodes) on a Log₁₀ scale for 367 assays in one plasma sample. A comparison is made between contacting the sample with a probe pool comprising all 367 assays and contacting the sample with the same set of probes divided into four abundance blocks. The counts for assays in Blocks A and B have increased significantly compared to the assays with lower counts when not using abundance blocks, allowing higher detection of the corresponding assays. Counts for Block D have correspondingly decreased, mitigating the loss of flow cell real estate, compared to the assays with higher counts when not using abundance blocks.

FIG. 4 shows the resulting counts (correctly paired barcodes) on a linear scale for 367 assays in one plasma sample. A comparison is made between contacting the sample with a probe pool comprising all 367 assays and contacting the sample with the same set of probes divided into four abundance blocks. The counts for assays in Blocks A and B have increased significantly compared to the assays with lower counts when not using abundance blocks, allowing higher detection of the corresponding assays. Counts for Block D have correspondingly decreased, mitigating the loss of flow cell real estate, compared to the assays with higher counts when not using abundance block.

FIG. 5 shows boxplots of the results in 54 plasma samples contacted with a probe pool of 372 assays divided into four abundance blocks and sorted by the median count within a block. The abundance blocks allow detection of wide ranges of protein abundance between the samples without sacrificing detection, or risk the lower ranges of a assay with high variation between samples falling below a robust detection of counts. The dashed line indicates 100 counts as a threshold for enough detection of counts.

### EXAMPLES

### Example 1—Exemplary Experimental Protocol

### Step 1—Sample Preparation and Incubation

16 Aliquots from each of 48 to 96 plasma samples are incubated with each of up to 16 proximity probe pools (four abundance blocks for each of four 384-probe pair panels) in 96-well or 384-well incubation plates.
Samples may be pre-diluted 1:10, 1:100, 1:1000 and 1:2000 for those probe pools containing assays that require it.
Dilution and dispensing of plasma samples into incubation solution can be performed manually, or by pipetting robot e.g. LabTech's Mosquito® HTS. Incubation solution is dispensed into the wells of the plate.
1 μl of sample is added to 3 μl of incubation mix at the bottom of each well, the plate is sealed with adhesive film, spun at 400×g for 1 minute at room temperature and incubated overnight at 4° C.
If using the above-mentioned pipetting robot, volumes may be decreased to 0.2 μl sample and 0.6 μl incubation mix (5× reduction).

The tables below give exemplary reagent formulations. Other components may be included, for example other blocking agents in the probe solutions.

TABLE 1
Sample Diluent and Negative Control Solution

| Component | Concentration | |
|---|---|---|
| NaCl | 8.01 | g/l |
| KCl | 0.2 | g/l |
| Na₂HPO₄ | 1.44 | g/l |
| KH₂PO₄ | 0.2 | g/l |
| BSA | 1 | g/l |

TABLE 2
Incubation Mix

| | 4 μl | 0.8 μl |
|---|---|---|
| | Incubation Volume | Incubation Volume |
| Reagent | Volume (μl) | Volume (μl) |
| Incubation Solution | 2.40 | 0.48 |
| Forward Probe Solution | 0.30 | 0.06 |
| Reverse Probe Solution | 0.30 | 0.06 |
| Sample | 1.00 | 0.20 |
| Total | 4.0 | 0.8 |

TABLE 3
Incubation Solution

| Component | Concentration | |
|---|---|---|
| Triton X-100 | 1.70 | g/l |
| NaCl | 8.01 | g/l |
| KCl | 0.2 | g/l |
| Na₂HPO₄ | 1.44 | g/l |
| KH₂PO₄ | 0.2 | g/l |
| EDTA Na-salt | 1.24 | g/l |
| BSA | 8.80 | g/l |
| Blocking-probes Mix | 0.199 | g/l |
| GFP | 1-5 | pM |

TABLE 4
Forward Probe Solution

| Component | Concentration | |
|---|---|---|
| NaCl | 8.01 | g/l |
| KCl | 0.2 | g/l |
| Na₂HPO₄ | 1.44 | g/l |
| KH₂PO₄ | 0.2 | g/l |
| EDTA Na-salt | 1.24 | g/l |
| Triton X-100 | 1 | g/l |
| BSA | 1 | g/l |

| | |
|---|---|
| Probes | 1-100 nM per probe |

TABLE 5
Reverse Probe Solution

| Component | Concentration | |
|---|---|---|
| NaCl | 8.01 | g/l |
| KCl | 0.2 | g/l |
| Na₂HPO₄ | 1.44 | g/l |
| KH₂PO₄ | 0.2 | g/l |
| EDTA Na-salt | 1.24 | g/l |
| Triton X-100 | 1 | g/l |
| BSA | 1 | g/l |
| Probes | 1-100 nM per probe | |
| Detection Control | 6.4-1188 | fM |
| Extension Control | 75-10686 | fM |

### Step 2—Proximity Extension and PCR1 Amplification

Extension and amplification are performed using Pwo DNA polymerase. PCR1 is performed using common primers for amplification of all extension products.

The incubation plate (from step 1) is brought to room temperature and centrifuged at 400×g for 1 minute. The extension mix (comprising ultrapure water, DMSO, Pwo DNA polymerase and PCR1 solution) is added to the plate, and the plate is then sealed, briefly vortexed and centrifuged at 400×g for 1 minute, then placed in a thermal cycler for the PEA reaction and preamplification (50° C. 20 min, 95° C. 5 min, (95° C. 30s, 54° C. 1 min, 60° C. 1 min) ×25 cycles, 10° C. hold). Preferably, a dispensing robot may be used to dispense the extension mix into the plate, e.g. the Thermo Scientific™ Multidrop™ Combi Reagent Dispenser. The forward common primer comprises the Illumina P5 sequencing adapter sequence (SEQ ID NO: 1).

TABLE 6
PCR1 Reaction Mix

| | 4 μl | 0.8 μl |
|---|---|---|
| | Incubation Volume | Incubation Volume |
| Reagent | Volume (μl) | Volume (μl) |
| MilliQ water | 75.0 | 15.00 |
| DMSO (100%) | 10.0 | 2.00 |
| PCR1 solution | 10.0 | 2.0 |
| DNA Polymerase (1-10 U/μl) | 1.0 | 0.2 |
| Incubation mix | 4.0 | 0.8 |
| Total | 100.0 | 20.0 |

TABLE 7
PCR1 Solution

| Component | Concentration | |
|---|---|---|
| Tris base | 168.40 | mM |
| Tris-HCl | 31.47 | mM |
| MgCl₂ hexahydrate | 10.00 | mM |
| dATP | 2.00 | mM |
| dCTP | 2.00 | mM |
| dGTP | 2.00 | mM |
| dTTP | 2.00 | mM |
| Forward “P5” primer | 10.00 | μM |
| Reverse primer | 10.00 | μM |

### Step 3—Pooling Abundance Blocks

PCR1 products from each of the four abundance blocks from a 384-probe pair panel are pooled together. This results in up to four PCR1 pools per sample, one for each 384-probe pair panel.

Different volumes can be taken from each block to even out the relative levels of assays between the blocks. Pooling of PCR1 products can be performed manually, or by pipetting robot.

### Step 4—PCR2 Indexing

A primer plate containing 48 to 96 reverse primers is provided (generally one primer in each well of a 96-well plate). Each reverse primer comprises the “IIlumina P7” sequencing adapter sequence (SEQ ID NO: 2) and a sample index barcode. A unique barcode sequence is used for PCR1 products from each different sample. Preferably each of the up to four PCR1 pools comprising the same plasma sample (one for each 384-probe pair panel) receive the same sample index, for easy identification and data processing. A forward common primer comprising the “IIlumina P5” sequencing adapter sequence (the same forward primer as used in PCR1) is provided in the PCR2 solution.

Each PCR1 pool is contacted with PCR2 solution containing the forward common primer, a single reverse (sample index) primer from the primer plate, and a DNA polymerase (Taq or Pwo DNA polymerase). Amplification is performed by PCR until primer depletion (95° C. 3 min, (95° C. 30 s, 68° C. 1 min)×10 cycles, 10° C. hold).

The theoretical end concentration of pooled PCR1 product is 1 μM (all primers used). PCR1 amplicons are diluted 1:20 dilution for PCR2, giving a starting concentration of 50 nM in each PCR2 reaction. The concentration of each PCR2 primer is 500 nM. PCR2 primer depletion should therefore occur after 3.3 cycles (10-fold amplification).

TABLE 8
PCR2 Reaction Mix

| Reagent | Volume (μl) |
|---|---|
| MilliQ water | 14.96 |
| PCR2 solution | 2.0 |
| DNA Polymerase (1-10 U/μl) | 0.04 |
| Sample index primer solution | 2.0 |
| Pooled PCR1 reactions | 1.0 |
| Total | 20.0 |

TABLE 9
PCR2 Solution

| Component | Concentration | |
|---|---|---|
| Tris base | 168.40 | mM |
| Tris-HCl | 31.47 | mM |
| MgCl₂ hexahydrate | 10.00 | mM |
| dATP | 2.00 | mM |
| dCTP | 2.00 | mM |
| dGTP | 2.00 | mM |
| dTTP | 2.00 | mM |
| Forward “P5” Primer | 5.00 | μM |

TABLE 10
Sample Index Primer Solution

| Component | Concentration | |
|---|---|---|
| Tris base | 1.948 | mM |
| Tris-HCl | 8.052 | mM |
| EDTA | 1 | mM |
| Sample index “P7” primer | 5.00 | μM |

### Step 5—End Pool

All 48 to 96 indexed sample pools belonging to the same 384-probe pair panel are pooled together, adding the same volume from each sample. This yields up to four final pools (or libraries), one for each 384-probe pair panel.

### Step 6—Purification and Quantification (Optional)

The libraries are purified separately using magnetic beads, and purified libraries' total DNA concentration is determined using qPCR with a DNA standard curve. AMPure XP beads (Beckman Coulter, USA), which preferentially bind longer DNA fragments, may be used in accordance with the manufacturer's protocol. The AMPure XP beads bind the long PCR products but do not bind short primers, thus enabling purification of the PCR product from any remaining primers.

Depletion of the PCR2 primers means that this purification step may not be necessary.

### Step 7—Quality Control (Optional)

A small aliquot of each (purified) library is analysed on an Agilent Bioanalyser (Agilent, USA), in accordance with the manufacturer's instructions, to confirm successful DNA amplification.

### Step 8—Sequencing

Libraries are sequenced using an Illumina platform (e.g. the NoveSeq platform). Each of the up to four libraries (from each 384-probe pair panel) is run in a separate “lane” of a flow cell. Depending on the size and model of flow cell and sequencer used, the up to four libraries may be sequenced in parallel or sequentially (one after the other) in different flow cells.

### Step 9—Data Output

Barcode (from each reporter nucleic acid molecule) and sample index (from the sample index primers) sequences are identified in the data, counted, summed and aligned/labeled according to a known barcode-assay-sample key.
“Matching barcodes” represent interactions between two paired PEA probes. The count is relative to the number of interactions in the PEA.
Counts for each assay and sample must be normalised using the internal reference controls to be able to compare between samples.
Each of the four abundance blocks has its own internal reference control.

Each 384-probe pair panel is separated based on the lane it is read out in. Each panel comprises the same 96 sample indexes and the same 384 barcode combinations and internal reference controls.

### Example 2—PEA with and without Abundance Blocks

A multiplex PEA was performed (using probes comprising antibodies conjugated to nucleic acid domains having the structure described in Version 6, above) to detect 367 proteins in plasma samples. Each probe contained a unique barcode sequence. A proximity probe pool comprising all 367 assays was incubated with the samples, and as a comparison, 4 aliquots from each of the plasma samples were incubated with each of 4 proximity probe pools (four abundance blocks comprising the 367 assays) in 96-well or 384-well incubation plates.

The PEA was performed as described above, except Step 3 was omitted for the proximity probe pool without abundance blocks. During amplification of the extension products, P5 and P7 sequencing adapters were added to each end of the products, along with a unique sample index for reporter nucleic acids from each different sample, and all extension products sequenced by massively parallel DNA sequencing, employing the reversible dye terminator sequencing technique using an Illumina NovaSeq platform. The extension product resulting from the probe pool with 367 assays and extension products resulting from the pooled abundance blocks totaling 367 assays were sequenced at separate times in separate flow cells.

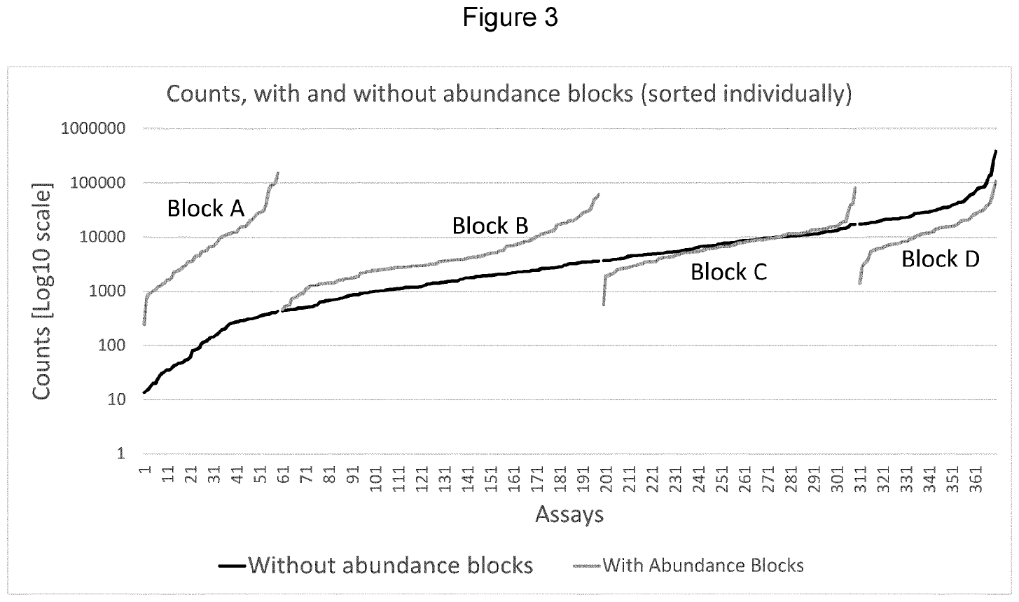

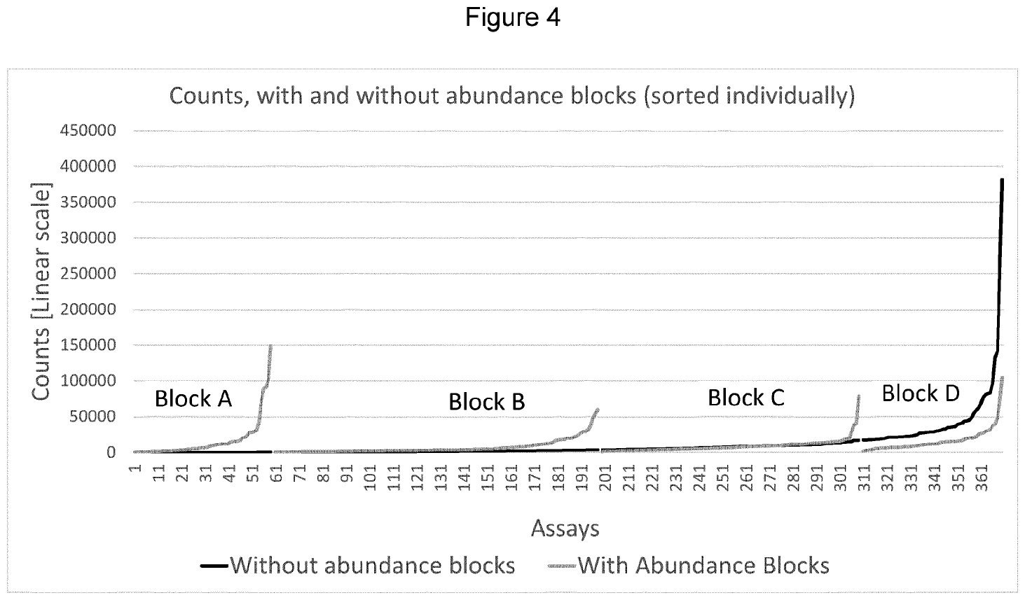

Results for one of the plasma samples can be seen in FIG. 3 and FIG. 4. The table below shows the ratio between the highest assay (counts) and lowest assay (counts) with and without using abundance blocks in the same plasma sample. The ratios in the abundance blocks are significantly lower than the ratio of the full pool of 367 assays, meaning the readouts of these assays use the flow cell real estate in a more optimal way (more counts for low abundance assays, fewer counts for high abundance assays).

| | | Number | Highest | Lowest | |
|---|---|---|---|---|---|
| | | of | Assay | Assay | High/Low |
| | | Assays | Count | Count | ratio |
| Pool | | 367 | 381 749 | 13 | 28 441 |
| Abundance | A | 59 | 149 065 | 243 | 614 |
| Blocks | B | 138 | 60 007 | 456 | 132 |
| | C | 110 | 78 685 | 563 | 140 |
| | D | 60 | 104 851 | 1394 | 75 |

### Example 3—PEA with Abundance Blocks on Samples with Assays of Varying Abundance

A multiplex PEA was performed (using probes comprising antibodies conjugated to nucleic acid domains having the structure described in Version 6, above) to detect 372 proteins in 54 plasma samples. Each probe contained a unique barcode sequence. 4 aliquots from each of the plasma samples were incubated with each of 4 proximity probe pools (four abundance blocks comprising the 372 assays) in 96-well or 384-well incubation plates.

The PEA was performed as described above. During amplification of the extension products, P5 and P7 sequencing adapters were added to each end of the products, along with a unique sample index for reporter nucleic acids from each different sample, and all extension products sequenced by massively parallel DNA sequencing, employing reversible dye terminator sequencing technique using an Illumina NovaSeq platform.

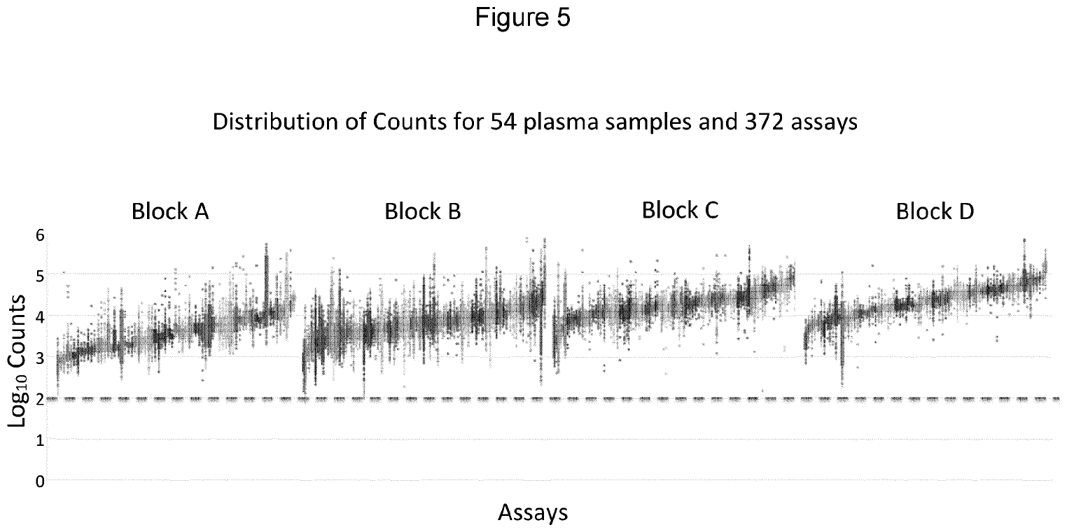

The results in FIG. 5 show that protein targets with a wide abundance range can be detected in the samples, without sacrificing the lower ranges of proteins with high variation in samples, or assays with relatively low abundance over all 54 samples, due to signal decrease (counts below a robust amount. e.g. 100 counts).

1. A method of detecting multiple analytes in a sample, wherein said analytes have varying levels of abundance in the sample, said method comprising:
(i) providing multiple aliquots from the sample; and
(ii) in each aliquot, detecting a different subset of the analytes by performing a separate multiplex assay for each aliquot, wherein the analytes in each subset are selected based on their predicted abundance in the sample.

2. The method of claim 1, wherein the analyte is a non-nucleic acid analyte.

3. The method of claim 1 or 2, wherein the analyte is or comprises a protein.

4. The method of any one of claims 1 to 3, wherein in each aliquot the analytes are detected by detecting a reporter nucleic acid molecule specific for each analyte.

5. The method of claim 4, wherein the reporter nucleic acid molecules are generated in the multiplex detection assay performed for each aliquot.

6. The method of claim 4 or 5, wherein the reporter nucleic acid molecules are amplified by PCR, and preferably are detected by nucleic acid sequencing.

7. The method of claim 6, wherein one or more adapters for sequencing are added to the reporter nucleic acid molecules in one or more amplification and/or ligation steps.

8. The method of claim 6 or 7, wherein the reporter nucleic acid molecules are subjected to at least a first PCR reaction to add at least a first adaptor for nucleic acid sequencing.

9. The method of claim 8, wherein the PCR products from the first PCR reaction are subjected to a second PCR reaction to add a second adaptor for nucleic acid sequencing.

10. The method of any one of claims 6 to 9, wherein at least one PCR reaction is run to saturation.

11. The method of any one of claims 1 to 10, wherein the reaction products of the separate multiplex assays or, where said reaction products are nucleic acid molecules, amplification products thereof, are pooled to create a first pool, and are amplified in the first pool.

12. The method of claim 11, wherein the reaction products of the multiplex assays are reporter nucleic acid molecules, and the method comprises:
amplifying the reporter nucleic acid molecules in first PCR reactions performed separately on each individual aliquot to generate first PCR products, pooling the first PCR products from individual aliquots to create a first pool, and performing a second PCR reaction on the first pool.

13. The method of claim 11 or 12, wherein different amounts of the reaction products or amplification products thereof are added to the first pool.

14. The method of any one of claims 11 to 13, wherein the method is performed in parallel for multiple different samples separately to generate reaction products, or amplification products thereof, for each sample, and wherein for each sample a separate first pool is created and a sample index is added to the products in the first pool by an amplification and/or ligation reaction.

15. The method of claim 14, wherein the separate first pool created for each sample comprises first PCR products, and wherein a sample index is added to the first PCR products in the second PCR reaction which is performed on the first pool for each sample.

16. The method of claim 14 or 15, wherein the indexed first pools generated for each sample are pooled together to create a second pool for performing nucleic acid sequencing.

17. The method of any one of claims 6 to 16, wherein the PCR reaction comprises an internal control for each aliquot.

18. The method of any one of claims 4 to 17, wherein the reporter nucleic acid molecule is generated in a proximity probe detection assay, in particular a proximity extension assay (PEA).

19. The method of any one of claims 4 to 16, wherein the reporter nucleic acid molecule comprises at least one barcode sequence, and detection of the reporter nucleic acid molecule comprises detecting the at least one barcode sequence, optionally in conjunction with a sample index,
preferably wherein the reporter nucleic acid molecule comprises a combination of barcode sequences from the nucleic acid domains of a pair of proximity probes, and detection of the reporter nucleic acid molecule comprises detection of the combination of barcode sequences.

20. The method of any one of claims 1 to 19, wherein the sample is a plasma or serum sample.

21. The method of any one of claims 18 to 20, wherein the analytes are detected using pairs of proximity probes, each proximity probe comprising:
(i) an analyte-binding domain specific for an analyte; and
(ii) a nucleic acid domain,
wherein both probes within each pair comprise analyte-binding domains specific for the same analyte, and each probe pair is specific for a different analyte, and wherein each probe pair is designed such that on proximal binding of the pair of proximity probes to their respective analyte the nucleic acid domains of the proximity probes interact to generate a reporter nucleic acid molecule;
wherein at least 2 panels of proximity probe pairs are used, each panel being for the detection of a different group of analytes, and for each panel separate aliquots of the sample are provided for the detection of a different subset of the analytes in the group; and
wherein (a) within each panel, every probe pair comprises a different pair of nucleic acid domains; and (b) in different panels the probe pairs comprise the same pairs of nucleic acid domains.

22. The method of claim 21, for detecting analytes from different samples, wherein the PCR products generated by amplification of the reporter nucleic acid molecules generated for each sample are provided with a sample index;
and wherein the PCR products generated from each different sample using the same panel of proximity probe pairs are pooled into a panel pool for nucleic acid sequencing, the PCR products generated using each panel being pooled into separate panel pools;
and wherein each panel pool is sequenced separately.

23. The method of any one of claims 7 to 22, wherein said nucleic acid sequencing is massively parallel DNA sequencing.
